(19) United States
(12) Patent Application Publication
(10) Pub. No.: US 2021/0177265 A1
(43) Pub. Date: Jun. 17, 2021

(54) SYSTEMS AND METHODS FOR IMAGING AND THERAPY SUITABLE FOR USE IN THE CARDIOVASCULAR SYSTEM

(71) Applicant: The General Hospital Corporation, Boston, MA (US)

(72) Inventors: Serguei Melnitochouk, Newton, MA (US); Guillermo J. Tearney, Cambridge, MA (US); Grace E. Baldwin, Weston, CT (US); Joseph A. Gardecki, Acton, MA (US)

(21) Appl. No.: 16/758,965

(22) PCT Filed: Oct. 25, 2018
(86) PCT No.: PCT/US2018/057584

§ 371 Date: Nov. 23, 2020

Related U.S. Application Data
(60) Provisional application No. 62/577,042, filed on Oct. 25, 2017.

(51) Int. Cl. A61B 5/00

(52) U.S. Cl. CPC A61B 5/0084; A61B 5/0044; A61B 5/0062; A61B 5/0075

(57) ABSTRACT

Probes, catheters, systems, methods, and ferrofluids are provides for acquiring direct visualization medical images of internal structures. The probes can include a channel, an optical waveguide, and a ferrofluid attractor. The ferrofluid attractor can be configured to magnetically attract the ferrofluid when exiting a distal port of the channel The medical image can be acquired through the ferrofluid, which is excluding blood from the area that the ferrofluid is occupying. The ferrofluid has a lower optical absorbance than blood, so the acquiring the image through the ferrofluid rather than through blood provides improved images.

### CROSS-REFERENCES TO RELATED APPLICATIONS

This application is based on, claims the benefit of, and claims priority to U.S. Provisional Application No. 62/577,042, filed Oct. 25, 2017, which is hereby incorporated herein by reference in its entirety for all purposes.

### STATEMENT REGARDING FEDERALLY SPONSORED RESEARCH

Not Applicable.

### BACKGROUND

The present disclosure relates to imaging and therapy methods, apparatuses, and devices, and more particularly to exemplary aspects of imaging and/or therapy methods and systems which can be suitable for use in visualizing and conducting therapy on a heart, valves, or blood vessels to obtain a diagnosis, acquire tissue, treat via the removal of pathology, or assist in the deployment of other devices.

Direct visualization of structures is often very useful for diagnosis and therapeutic interventions in medicine and surgery. However, physicians currently do not have a reliable means to directly visualize structures inside the beating heart and its chambers, or inside the major blood vessels. This is due to light being attenuated by blood.

Fluoroscopy and echocardiography are currently available modalities for real-time imaging of cardiovascular structures. While these techniques can be used to guide certain minimally invasive intracardiac procedures, but both are indirect and imprecise. This tends to make such procedures time and resource consuming. In addition to significantly prolonged procedural times, fluoroscopy is associated with risks. For example, fluoroscopy exposes patients and the clinical team to significant ionizing radiation. As another example, transesophageal echocardiography (TEE) is associated with a risk of esophageal injury. As yet another example, there is a risk of an inadvertent tissue injury or perforation by a device or a catheter that is guided by fluoroscopy or TEE during indirect visual navigation through the heart chambers or great vessels that is exacerbated by the relatively poor quality of the visualization.

Only a few methods have been attempted to directly visualize structures inside the heart, but none have found a widespread acceptance in clinical practice. For example, direct contact between an endoscope and cardiac tissue can provide a visualization, but such a visualization shows only an extremely small field. As another example, use of a transparent toroidal balloon chamber that displaces blood between the lens and the object could facilitate visualization, but does not allow any instrumentation through the balloon itself. As yet another example, displacement of blood with pressurized transparent fluid boluses is not capable of maintain imaging for sustained periods of time, and can cause hemodynamic instability. Finally, a complete replacement of intracardiac blood with a transparent nourishing perfusate can facilitate imaging, but also requires utilization of peripheral cardiopulmonary bypass and comes with substantial cost in addition to being a substantially more invasive procedure than fluoroscopy.

Therefore, the current unmet need lies in the absence of a method and apparatus for a direct imaging of the endocardial surface of the heart and blood vessels, also known herein as cardioscopy. In addition to diagnostic direct visualization, physicians also need a means for obtaining tissue, biopsy, and/or tissue removal of any pathology on a beating heart and blood vessels. Further therapeutic applications arise from a unique benefit of direct visualization of the intracardiac or intravascular structures in either energy delivery during ablative procedures or during delivery and deployment of various medical devices inside the heart or great vessels. All current diagnostic and therapeutic interventions on the heart and great vessels would potentially benefit from a radically new and direct imaging modality.

There are numerous examples of limitations of current imaging modalities in clinical practice. In case of massive or sub-massive pulmonary embolism (PE), currently available options (e.g., systemic thrombolysis, catheter-based direct thrombosis, and angiovac aspiration) have various drawings. For example, system thrombosis is very non-specific and frequently ineffective. As another example, catheter-based direct thrombolysis does not physically remove a large burden of the remaining clot. As yet another example, angiovac aspiration systems generally lack direct visualization of the thrombus, and procedures using such systems often rely instead on fluoroscopy, resulting in a very imprecise aspiration of the clot, in addition to requiring an invasive veno-venous bypass circuit. Ultimately, currently employed surgical embolectomy via sternotomy on a heart-lung machine is extremely invasive and requires substantial postoperative recovery. PE is very common and a massive or sub-massive PE is very morbid and frequently fatal. It is not surprising, therefore, that virtually all currently available therapeutic interventions targeted at PE are associated with substantial periprocedural morbidity and mortality either due to its significant ineffectiveness or a very radical invasiveness.

Further limitations include lack of direct visualization of the endocardial surface during ablative procedures for various arrhythmias, such as atrial fibrillation, supraventricular tachycardia, or ventricular tachycardia. Currently available options include employment of the catheter systems that deliver energy to create a tissue scar, thus interrupting micro or macro-reentrant circuits. The procedures tend to be long and frustrating because of lack of direct visualization of the catheter in relation to the endocardial surface and anatomical structures. Frequently fluoroscopy, transesophageal echocardiography, and/or intracardiac echocardiography are employed to get the task accomplished. However, despite all currently available indirect imaging modalities, most ablation procedures are frequently ineffective and require repeat interventions. The task would be much easier accomplished with a direct visualization of the catheter in the intracardiac chamber.

Another example is how heart transplant patients currently undergo multiple myocardial biopsies as part of their organ rejection surveillance regimen. Currently, a bioptome is advanced blindly under fluoroscopy guidance through the tricuspid valve. Not surprisingly, as the result of multiple biopsy sessions and blind passages across the tricuspid valve, the leaflets of the tricuspid valve are frequently injured and destroyed leading to a subsequent severe tricuspid regurgitation. Sometimes these very sick patients have to undergo either a heart re-transplant or a very high-risk tricuspid valve replacement via open heart surgery due to potentially avoidable injury of the tricuspid valve. Direct visualization of the bioptome passing across the tricuspid valve would ensure less injury to the tricuspid valve and make biopsy procedure more effective and less time consuming. The same can be said about guidance of the bioptome for biopsy of other intracardiac or intravascular pathologies.

However, perhaps most clinically relevant and time-pressing is the current suboptimal visualization modality in deployment of various currently available intracardiac or intravascular devices and related procedures, including, but not limited to, transcatheter aortic valve replacement, left atrial appendage occlusion, chronic total occlusion, transcatheter mitral valve repair, patent foramen ovale closure, transcatheter pulmonary valve replacement, paravalvular leak closure, and percutaneous transluminal coronary angioplasty. One example of percutaneous tricuspid or mitral annuloplasty devices, the currently available strategy employs both, fluoroscopy and echocardiography guidance to deploy and secure these devices around the valve hopefully well in the annular tissue. However, the platform is quite risky because of the neighboring coronary arteries, conduction system, and other anatomical structures. Due to the indirect imaging provided by fluoroscopy and echocardiography, the deployment is imprecise, time-consuming, and carries a high risk of injuring a neighboring anatomical structure, such as a coronary artery, conduction system, valve itself, or other anatomical structure. Further, the annulus of the valve, tricuspid or mitral, is a very thin structure and is best identified by its whitish colored line between atrial wall and actual valve leaflet tissue. The precision that is required to place an annuloplasty device into the annular tissue of the valve can be best achieved only by a direct visualization of the anatomical structure and not so much by a current guesswork-based on indirect and imprecise fluoroscopy and echocardiography. The current platform of indirect guidance by fluoroscopy or echocardiography is a far cry from what a proceduralist would prefer in terms of image quality to deploy a needed device.

Direct visualization and guidance in the delivery and deployment of various medical devices in the field of heart and vascular disease would provide a more successful, more durable, more precise, less time-consuming, and less complications-prone platform. It would literally revolutionize the way the numerous devices are placed in the heart and/or in the great vessels.

### BRIEF SUMMARY

In an aspect, the present disclosure provides a probe. The probe includes a proximal portion and a distal portion. The probe includes a channel, an optical waveguide, and a ferrofluid attractor. The channel has a proximal port, a distal port, and an interior surface. The proximal port is positioned at the proximal end of the probe. The distal port is positioned at the distal portion of the probe. The interior surface is composed of a material that is chemically and magnetically inert to a ferrofluid. The channel, the proximal port, and the distal port have size dimensions that allow the ferrofluid to enter the channel via the proximal port, move along the channel, and exit the channel via the distal port when the ferrofluid is introduced at a predefined pressure. The optical waveguide has a proximal waveguide end and a distal waveguide end. The proximal waveguide end is positioned at the proximal portion of the probe. The distal waveguide end is positioned at the distal portion of the probe. The ferrofluid attractor is coupled to the distal end of the probe. The ferrofluid attractor has magnetic properties and positioning relative to the distal port to magnetically attract the ferrofluid when exiting the distal port.

In another aspect, the present disclosure provides a catheter. The catheter includes a probe as described herein and a sheath configured to receive the probe.

In a further aspect, the present disclosure provides an optical imaging system. The optical imaging system includes an optical imaging light source, an optical imaging detector, a probe as described herein, a circulator, and an optical imaging controller. The circulator is coupled to the optical imaging light source, the optical imaging detector, and the optical waveguide. The circulator is configured to direct light from the optical imaging light source to the optical waveguide and from the optical waveguide to the optical imaging detector. The optical imaging controller is coupled to the optical imaging detector and configured to provide an optical imaging signal output representative of an optical signal measured at the optical imaging detector.

In yet another aspect, the present disclosure provides an optical coherence tomography (OCT) system. The OCT system includes an OCT light source, an OCT detector, a probe as described herein, a circulator, and an OCT controller. The circulator is coupled to the OCT light source, the OCT detector, and the optical waveguide. The circulator is configured to direct light from the OCT light source to optical waveguide and from the optical waveguide to the OCT spectrometer. The OCT controller is coupled to the OCT spectrometer and configured to provide an OCT signal output representative of an OCT signal measured at the OCT spectrometer.

In yet a further aspect, the present disclosure provides a ferrofluid for use in direct visualization medical imaging of an internal structure. The ferrofluid includes ferromagnetic particles and a biologically inert solvent. The ferromagnetic particles are present in an amount by weight of between 0.1 milligrams of iron per milliliter and 100 milligrams of iron per milliliter.

In another aspect, the present disclosure provides a method of acquiring a direct visualization medical image of an internal structure. The method includes: a) introducing a ferrofluid into an area near the internal structure, thereby displacing a biological fluid within the area, the ferrofluid retained in the area using a magnetic effect; and b) acquiring the direct visualization medical image of the internal structure through the ferrofluid.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF DRAWINGS

The invention will be better understood and features, aspects and advantages other than those set forth above will become apparent when consideration is given to the following detailed description thereof. Such detailed description makes reference to the following drawings.

FIG. 1 is a schematic of a probe, in accordance with an aspect of the present disclosure.

FIG. 2 is another schematic of a probe, in accordance with an aspect of the present disclosure.

FIG. 3 is a schematic of a use of a probe, in accordance with an aspect of the present disclosure.

FIG. 4 is another schematic of a use of a probe, in accordance with an aspect of the present disclosure.

FIG. 5 is an absorbance spectrum of the ferrofluid prepared in Example 1.

FIG. 6 is an image of a ferrofluid cloud formed in a buffer solution, as described in Example 2.

FIG. 7 is an image of a ferrofluid cloud formed in whole blood, as described in Example 3.

FIGS. 8A to 8C are various images taken with and without a ferrofluid cloud, as described in Example 4.

FIG. 9A is yet another schematic of a probe, in accordance with an aspect of the present disclosure.

FIG. 9B is still another schematic of a probe, in accordance with an aspect of the present disclosure.

FIGS. 10A1 to 10B3 are various schematics of probes, in accordance with aspects of the present disclosure.

FIGS. 11A and 11B are additional schematics of probes, in accordance with aspects of the present disclosure.

FIG. 12 is a cross-section schematic of a probe, in accordance with an aspect of the present disclosure.

FIG. 13 is a photograph of a probe, in accordance with an aspect of the present disclosure.

FIGS. 14A to 14I are cross-sections of various magnet configurations in accordance with aspects of the present disclosure.

FIGS. 15A to 15E are additional magnet configurations in accordance with aspects of the present disclosure.

FIG. 16A is various magnet configurations and corresponding magnetic flux models in accordance with aspects of the present disclosure.

FIG. 16B is a depiction of flux density for magnet configurations of FIG. 16A in accordance with aspects of the present disclosure.

FIG. 17 is a magnetic flux model corresponding to a particular magnet configuration of FIG. 16A in accordance with an aspect of the present disclosure.

FIG. 18 is an absorbance spectrum of Feraheme at various concentrations.

FIG. 19 is an absorbance spectrum of two different ferrofluids at various concentrations.

FIG. 20 is a depiction of wavelength spectra for different filters and corresponding ferrofluid guided images.

FIGS. 21A to 21D is a series of images taken with and without ferrofluid guided imaging in a pulsatile pump system, as described in Example 9.

FIGS. 22A and 22B are a series of images taken using ferrofluid guided imaging in a sheep heart, as described in Example 10.

FIG. 23 is a series of images showing the use of a bioptome by ferrofluid guided imaging, as described in Example 11.

FIG. 24 is a photograph of a pulsatile pump system used for testing, as described in Example 12.

FIG. 25 is a photograph of a probe implemented in accordance with an aspect of the present disclosure, as described in Example 13.

FIG. 26 is a photograph of a probe implemented in accordance with an aspect of the present disclosure, as described in Example 14.

FIGS. 27A to 27E is a series of images taken using ferrofluid guided imaging in a narrow tube simulating a coronary artery in accordance with an aspect of the present disclosure, as described in Example 15.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

Numeric ranges disclosed herein are inclusive of their endpoints. For example, a numeric range of between 1 and 10 includes the values 1 and 10. When a series of numeric ranges are disclosed for a given value, the present disclosure expressly contemplates ranges including all combinations of the upper and lower bounds of those ranges. For example, a numeric range of between 1 and 10 or between 2 and 9 is intended to include the numeric ranges of between 1 and 9 and between 2 and 10.

Lengths and distances described herein are described in terms of optical path length lengths and distances, unless the context clearly dictates otherwise. Accordingly, light traveling along a coiled optical fiber travels a distance that is equal to the uncoiled length of the optical fiber, not the physical distance between the input and output of the optical fiber.

As used herein, the term “substantially-transparent” refers to the ability to successfully transmit light through a medium. “Substantially” referring to the fact that the medium is neither optically transparent nor completely absorbent. For example, a medium that is substantially transparent would allow visualization of a target image with a light based imaging device at a resolution that allows desired structures to be discernable. It is assumed that a substantially transparent medium will have an absorbance at minimum less than blood, allowing light transmittance at a depth and resolution necessary for the specific application. The present disclosure provides systems and methods that have a variety of advantages relative to those available in the art. The following description of these advantages is not intended to be limiting, nor is it intended to imply that the systems and methods can only be used to achieve these advantages.

For example, the present disclosure provides examples of probes, catheters, optical systems, OCT systems, ferrofluids, and processes as described herein. Features described in connection with one or more of aspects of these examples are generally applicable to the others. For example, features described in connection with probes are generally applicable to OCT systems, and features described in connection with ferrofluids are generally applicable to the processes.

In some aspects, mechanisms described herein can be used (e.g., by physicians) to directly image and conduct minimally-invasive therapy on cardiovascular structures in the presence of flowing blood. For example, a device for directly imaging cardiovascular structures can include a flexible cardioscope (e.g., an endoscope used in the cardiovascular system) that includes a magnetic tip. In such an example, a liquid that becomes magnetized when placed in the presence of a magnetic field (sometimes referred to herein as a ferrofluid), is injected through a working channel to the tip of the cardioscope. The magnetic field produced by the magnetic tip of the cardioscope in such an example can cause the ferrofluid to localize near the tip of the cardioscope scope, which can form a ferrofluid cloud that displaces blood. In such an example, light can more easily penetrate through the ferrofluid cloud than through blood, which can facilitate direct visualization of a target. Additionally, a minimally-invasive surgical procedure can be conducted through the ferrofluid cloud, while the target is continuously visualized. After such a procedure is over, suction through the working channel can be used to remove the ferrofluid from the tip of the cardioscope.

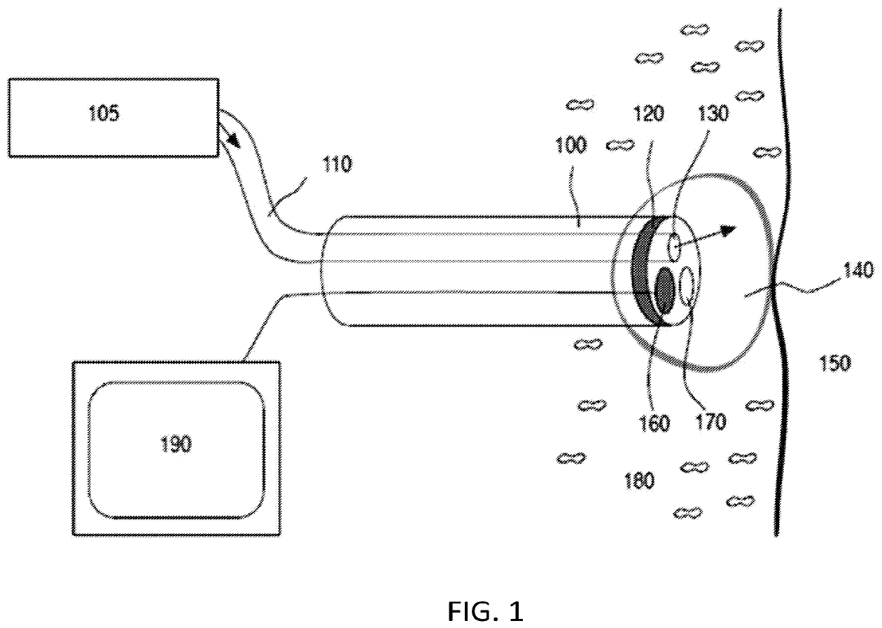

As described above, one solution for direct cardioscopy entails a substantially transparent ferrofluid that displaces blood while staying around a magnetized tip of a flexible probe or cardioscope (see, e.g., FIG. 1). Ferrofluids are conventionally a colloidal liquid made of ferromagnetic particles in a carrier fluid that become magnetized in the presence of a magnetic field. An aspect of this disclosure is a substantially transparent ferrofluid that can displace blood and through which an optical spectroscopic measurement or image can be acquired. In one exemplary aspect of this disclosure, a cardioscope can be advanced into the heart or into the pulmonary artery in cases of pulmonary embolism using any suitable technique or combination of techniques (e.g., transaortic, transapical, transfemoral, transeptal, transradial, transfemoral, transsubclavian, transjugular, etc.). The substantially transparent ferrofluid would then emanate from the tip of the endoscope via a separate lumen within the probe and remain at least partially adjacent to the probe as a spherical “cloud” around the tip of the probe, while displacing the blood in between the cardiovascular anatomical structure and the imaging arrangement within the cardioscope. For cases where a direct biopsy is required, bioptome forceps may be advanced via a lumen within the cardioscope probe and the structure of interest is biopsied under direct visualization through the ferrofluid cloud. In cases of the need to aspirate (e.g., clot or vegetation, etc.), the tip of the cardioscope is pushed against the clot or vegetation, the ferrofluid is aspirated back, and then the clot or vegetation is aspirated via a lumen within the cardioscope. A basket can be deployed through the ferrofluid behind the clot to assist in clot removal.

The systems and methods described herein can be utilized in multiple clinical applications in cardiology, and can facilitate expansion of the field of minimally-invasive cardiovascular surgery. For example, the mechanisms described herein can be used in connection with diagnosis and/or treatment of atrial fibrillation, pulmonary embolism, heart valve disease, heart failure, coronary artery disease, conduction disorders, vascular disease, etc. As another example, the mechanisms described herein can facilitate direct imaging of the heart and endovascular structures, which can be used by a healthcare provider (e.g., a physician) during various procedures. For example, the physician can use the mechanisms described herein to perform directly visualized biopsy and tissue removal procedures. As another example, the physician can use the mechanisms described herein to more effectively extract and/or aspirate clots. As yet another example, the physician can use the mechanisms described herein to more efficiently perform ablation procedures. As still another example, the physician can use the mechanisms described herein to provide visual guidance during deployment of various intracardiac and endovascular devices. As a further example, the physician can use the mechanisms described herein to directly identify perivalvular leaks. As another further example, the physician can use the mechanisms described herein to perform numerous directly visualized intracardiac procedures such as atrial septectomy, stitch placement, and others. The mechanisms described herein can be used by various types of healthcare providers in a variety of settings. For example, the mechanisms described herein can be used by interventional cardiologists and cardiac surgeons performing procedures in hybrid operating rooms or cardiac catheterization labs.

In a more particular example, applications of this disclosure can include aspiration of blood clots from the pulmonary arteries in cases of pulmonary embolism. With the direct cardioscopy of this disclosure, the clot is identified in the pulmonary artery and then directly aspirated via a main lumen. This would allow a much more elegant, less expensive, less invasive, more expedited, and more thorough treatment of pulmonary embolism. Another application of the cardioscopy would include direct visualization of the endocardial surface during catheter ablation of either atrial fibrillation or ventricular tachycardia. Direct visualization would reduce risk of spontaneous and potentially very hazardous perforations that still occur during current fluoroscopic and echocardiographic guidance. It would also potentially improve the actual effectiveness of the ablation procedure due to a better and more precise localization of the catheter on the endocardial surface. Another application would include cardioscopic ferrofluid-guidance during placement of the miniaturized leadless pacemaker, which is currently placed under fluoroscopic guidance. Ferrofluid-guided cardioscopy would assist in a more precise and a less traumatic placement of the pacemaker device and potentially would reduce its future dislodgement or interaction with intracardiac structures such as valve or chordal tissue.

Other potential applications lie in the directly visualized endomyocardial biopsy of the right ventricle in the heart transplant patients. Having the means of directly visualized biopsies via this disclosure would obviate tricuspid valve injury.

Further applications include directly visualized biopsy capabilities of the entire spectrum of the right sided and left sided cardiac lesions—whether those are endocarditis vegetations or cardiac tumors. Also, ferrofluid cardioscopy would allow one to assist an interventionalist in deployment of numerous intracardiac devices, such as an Amplatzer device for ASD closure, Watchman device for the left atrial appendage occlusion, annuloplasty device on the tricuspid valve, MitraClip deployment on the mitral leaflets or Neochord placement on a flail mitral leaflet, as well as stent graft placement in the aorta, and so on. A robust ferrofluid cardioscopy device would enable numerous further developments of intracardiac interventions on a beating heart and blood vessels. The field of ferrofluid cardioscopy, in and of itself, is a completely unchartered territory with a practically unlimited spectrum of clinical applications.

In case of MitraClip deployment, one would have a much easier and quicker way to perform the transseptal puncture and to position the clip on the mitral leaflets. With the help of spherical fluid around the tip of the catheter providing direct visualization one would identify foramen ovale anatomy much more precise and quicker. One would assess the anatomy of anterior and posterior leaflets directly, one would identify the ruptured chords or other pathology, and one would much better identify the best location for the most successful placement of the MitraClip. This would obviate the need for a lengthy and frustrating guidance by the TEE and fluoroscopy and would significantly shorten procedural time while allowing a much easier and more satisfying placement of the device on the leaflets resulting in a much more durable and effective treatment.

A direct cardioscopy platform such as the one described herein would allow a much more precise placement of the annuloplasty ring and would ensure a higher success, shorter procedure time, and less injury of the neighboring structures. Overall, the benefit of a direct visualization during deployment of the intracardiac or intravascular devices is multifaceted and difficult to quantify.

With the present disclosure, the patient benefit lies in the less invasive approach, since open heart surgery is very invasive and carries significant associated morbidity and mortality. If there is a way to remove a clot from the pulmonary artery without opening the sternum and being placed on the heart lung machine, every patient would sign up for it. The clinician benefit lies in a more expedited, less invasive aspiration of the clot. Potentially, with the established technology, the ferrofluid cardioscopy procedure could be performed at the bedside, just like bronchoscopy or some other endoscopic procedure. The payer benefit of this disclosure lies in the less expensive treatment (both surgery and catheter-based thrombolysis or Vortex procedure need to be done in either an operating room or in an angio suite) and shorter hospital stay. With the direct ferrofluid cardioscopic aspiration of the clot, the procedure could potentially be done at the bedside and would involve only a percutaneous access via the femoral vein. Overall, percutaneous embolectomy by means of ferrofluid cardioscopy would be a significantly more elegant solution than currently existing alternatives.

Referring to FIG. 1, an exemplary schematic of a probe 100 is illustrated. The probe 100 of FIG. 1 is illustrated in a configuration that is optimized for use in imaging from an end surface of the probe. The probe 100 includes a channel 110. The probe 100 can be flexible. A container 105 contains a ferrofluid to be introduced and removed via the channel 110. The probe 100 includes a ferrofluid attractor 120 at a distal portion of the probe 100. The ferrofluid emerges from a distal port 130 or distal opening 130 located at a distal portion of the probe 100. When introduced via the channel 110 and the distal port 130, the ferrofluid forms a ferrofluidic cloud 140 due to being attracted by the ferrofluid attractor 120. The probe 100 is illustrated engaging a target 150 that will be imaged. The probe 100 includes an optional working channel 170, through which a medical device and/or apparatus can be introduced to the target 150. The probe 100 includes an optical waveguide 160 for coupling light to and from the target 150. In use, the ferrofluidic cloud 140 displaces surrounding biological fluid 180, thereby providing a medium of generally known optical properties (i.e., the ferrofluid) between the optical waveguide 160 and the target 150. A spectroscopic imaging device 190 is optically coupled to the optical waveguide 160 and is configured to acquire a spectroscopic image of the target 150 through the ferrofluidic cloud 140.

The channel 110 can have a proximal port (not illustrated) positioned at a proximal portion of the probe. The channel 110, the proximal port, and the distal port 130 can have size dimensions that allow the ferrofluid to enter the channel via the proximal port, move along the channel 110, and exit the channel 110 via the distal port 130 when the ferrofluid is introduced at a predefined pressure. The size dimensions also allow the opposite motion when suction is introduced to the proximal port at a predefined negative pressure. The channel 110 has an interior surface that can be a material that is chemically and magnetically inert to the ferrofluid.

The ferrofluid attractor 120 can attract the ferrofluid based on magnetic properties of the ferrofluidic attractor 120 and the ferrofluid, and can be implemented using various different materials that have various different magnetic properties. For example, the ferrofluid attractor 120 can include a permanent magnet component. In a more particular example, the ferrofluid attractor 120 can be a neodymium iron boron permanent magnet, a samarium cobalt permanent magnet, an alnico permanent magnet, a ceramic permanent magnet, and/or a ferrite permanent magnet. The ferrofluid attractor 120 can be a printed 3D magnet that is printed by a magnet 3D printer to more precisely control the position of the dipoles. As another example, the ferrofluid attractor 120 can include an electromagnet component. As yet another example, the ferrofluid attractor 120 can include a ferromagnetic (which may be generally unmagnetized), that has a magnetic susceptibility sufficient to magnetically attract a ferrofluid having a persisting ferromagnetism. A variety of different coatings can be used in connection with the ferrofluidic attractor 120, such as nickel, gold, chrome, copper, epoxy resin, zinc, Teflon, silver, etc., to prevent undesirable chemical interactions between the ferrofluidic attractor 120 and biological fluid 180 (or other components of the probe 100). The ferrofluid attractor 120 can be a single component (e.g., a single permanent magnet, a single electromagnet, a single ferromagnetic (but unmagnetized) component, etc.). In such an example, the ferrofluid attractor 120 can be monolithic. Alternatively, the ferrofluid attractor 120 can include multiple attractor components. For example, the ferrofluid attractor 120 can include a permanent magnet, and an electromagnet. As another example, the ferrofluid attractor 120 can include multiple permanent magnets that are arranged to provide a particular magnetic field strength and/or shape.

One or more magnetic properties of the ferrofluid attractor 120 can be tuned to be control how strongly the ferrofluid is magnetically attracted to the ferrofluid attractor 120. For example, the magnetism can be tuned to be strong enough to retain the ferrofluidic cloud 140 in a stable orientation despite the movement of surrounding fluid, such as the pumping of blood through a blood vessel. In a more particular example, the ferrofluid attractor 120 can be configured to transition between a state of relatively high magnetism and a state of low relatively low (or no) magnetism. For example, a magnet of the ferrofluid attractor 120 can be coupled to an actuator that is configured to move the magnet closer to, and farther from, the distal port(s) 130 and/or a surface of the probe 100, altering the magnetic field strength outside of probe 100. As another example, a magnetic component of the ferrofluid attractor 120 can be configured to have an adjustable magnetic field strength. In a more particular example, when a component of the ferrofluid attractor 120 is implemented as an electromagnet, a magnetic field strength can be controlled based on the amount of current passed through the electromagnet, based on a position of a core material (e.g., a ferromagnetic core) within a coil of the electromagnet, etc. Additionally or alternatively,

The ferrofluid attractor 120 can be modified to alter a shape of the magnetic field. For example, in the case of a toroidal-shaped magnet, the corners of the top of the magnet (i.e., the portion of the magnet closest to the distal port 130 can be covered by a material that reduces the magnetic attraction in that region, forcing the ferrofluid cloud toward the center axis of the probe 100 in a region of relatively less dense magnetic field lines. This is merely an example, and a similar technique can be utilized with differently shaped and sized magnets to alter the shape of the magnetic field of the ferrofluid attractor 120 and influencing the shape of the ferrofluidic cloud 140. Additionally, cohesion of the ferrofluidic cloud 140 with the surrounding biological fluid 180 can be used to collect the ferrofluid cloud 140 more densely at the center axis of the probe 100. The ferrofluid attractor 120 can extend beyond the probe 100 circumferentially to stabilize and concentrate the ferrofluidic cloud 140, while leaving an opening that still maintains the ability to direct light forward and/or to the side and utilize tools or suction. The ferrofluid attractor 120 can have an oscillating magnetic field direction, which can control a net movement of the ferrofluid in a manner that resists dissipation into flowing of the biological fluid 180 (e.g., blood and/or other solutions around the ferrofluid cloud 140). For example, a permanent magnet can be physically rotated to cause net movement of the ferrofluid cloud 140 which can be controlled based on the rotation of the permanent magnet. As another example, oscillation of the current through an electromagnet can cause net movement of the ferrofluid cloud 140 which can be controlled based on the frequency, amplitude, and/or magnitude of the current signal.

The target 150 can be an intracardiac structure, a blood vessel wall, cardiovascular tissue, skin, gastrointestinal tissue, lung tissue, brain tissue, urologic tissue, gynecologic tissue, a thrombus, cardiac vegetation, a certain pathology of interest, a foreign body, a medical device, or the like.

The working channel 170 can be configured to receive a medical instrument or other device for delivery to the distal portion of the probe 100. The medical instrument or other device can be a suction catheter, biopsy forceps, a clip, a stent, a blood clot retrieval basket, a tissue ablator, a hook, an ablation catheter, a retrieval basket, a brush, a fixation device (e.g., a screw), an annuloplasty device or the like, a small leadless catheter, or a combination thereof.

The present disclosure also provides catheters. The catheter can include a probe (e.g., the probe 100) as described herein and a sheath configured to receive the probe. The catheter can be of various diameters for different applications. The catheter can be an angioscope, cardioscope, endoscope, cardioscopic catheter, nasogastric tube, any laparoscopic imaging device, etc.

The present disclosure also provides optical imaging systems. The optical imaging systems can include an optical imaging light source, an optical imaging detector, a probe (e.g., the probe 100) as described herein, an optical circulator, and an optical imaging controller. The optical circulator is coupled to the optical imaging light source, the optical imaging detector, and an optical waveguide (e.g., the optical waveguide 160). The optical circulator can be configured to direct light from the optical imaging light source to the optical waveguide and from the optical waveguide to the optical imaging detector. The optical imaging controller can be coupled to the optical imaging detector and configured to provide an optical imaging signal output representative of an optical signal measured at the optical imaging detector. The optical imaging system can be a fluorescence, autofluorescence, Raman, OCT, SECM, or other spectroscopic imaging system. The optical light source and optical detector can be chosen for the appropriate type of spectroscopic imaging.

The present disclosure also provides OCT systems. The OCT systems includes an OCT light source, an OCT detector, a probe (e.g., the probe 100) as described herein, an optical circulator, and an OCT controller. The circulator is coupled to the OCT light source, the OCT detector, and an optical waveguide (e.g., the optical waveguide 160). The optical circulator can be configured to direct light from the OCT light source to the optical waveguide and from the optical waveguide to the OCT detector. The OCT controller can be coupled to the OCT detector and configured to provide an OCT signal output representative of an optical signal measured at the OCT detector. The OCT light source can be a broadband light source.

The present disclosure also provides ferrofluids for use in connection with the probes and systems described herein. The ferrofluids can be used for direct visualization medical imaging of an internal structure. The ferrofluids can include ferromagnetic particles (e.g., iron particles) and a biologically inert carrier fluid. Ferromagnetic particles present in an amount of 0.1 mg Fe/ml or less to as high as 100 mg Fe/ml is conceivable. Also, dosages ranging from less than 0.2 mg Fe/kg to as high as a single dose of 1000 mg Fe is conceivable. Specific dosage and concentration may vary based on the desired application and imaging device. Also, the ferromagnetic particle content (e.g., iron content) may be able to be higher, as limited by toxicity of the specific ferrofluid in the human body.

The ferromagnetic particles can include a coating. There are a wide range of conceivable coatings, and the specific coating may vary based on the specific application and imaging device. Possible carbohydrate coatings include dextran, galactose, mannose, glucose, ethylene glycol, citrate, fucose, carboxymaltose, carboxydextran, polyethylene glycol, carboxy-methyldextran, arabinogalactan, and poly-styrene, and the like. Other coatings include hydroxyphosphonate, folate, sodium ferric gluconate, silica, carboxylates, polyamidoamine, lipid bilayers, curcumin, hydrophilic polymers, hydrophobic polymers, polymers that are neither hydrophobic nor hydrophilic, amphiphilic ligands, and additional bound proteins that can be single amino acids or chains of amino acids, etc. Coatings with a range in weight from 1 kilodalton (kD) to 2000 kD are conceivable. Dextran, which is often used as a ferromagnetic nanoparticle coating, ranges from 3 to 2000 kD.

The ferromagnetic particles can be of a size that substantially reduces the amount of light that is scattered by the ferromagnetic particles. The ideal ferromagnetic particle size will differ on the application and the light based imaging device. For example, depending on the resolution or wavelength utilized in the light-based imaging device, different particles sizes will scatter more or less light. Particle coatings ranging from 6 to 100,000 nm is conceivable. Further, the use of superparamagnetic iron oxide particles (SPIO) which range from 100 to 200 nm, ultrasmall superparamagnetic iron oxide particles (USPIO) which are less than 50 nm, and micron sized particles of iron oxide (MPIO) which are greater than 1000 nm, are all conceivable.

The ferrofluid can include a viscosity enhancing agent. The viscosity enhancing agent can be present in as little as 1% or less of the solution, or as much as the saturation point of the solution. For example, for dextran the saturation point occurs roughly when the ratio of dextran to water is 2:1. Besides dextran, other viscosity enhancing agents can include any agent that is both water-soluble and non-toxic. Examples of which can include other polysaccharides or oligosaccharides, such as starch, glycogen, callose, chyrsolaminarin, xylan, arabinoxylan, mannan, fucoidan, hydroxyethyl cellulose, and galactomannan. In addition, biocompatible oils can be used as a viscosity enhancing agent for some clinical applications.

Ferrofluid with a viscosity between 0.089 centipoise (cP) to 10,000 cP is conceivable. In certain applications, the viscosity can be between 3 to 10 cP, which is near the viscosity of blood.

The ferrofluid can be substantially transparent. The ferrofluid can have an average optical absorbance greater than water and less than blood for at least one wavelength between 400 and 1400 nm. The specific wavelength and transparency can vary based on the clinical application and imaging device, and can be related to the concentration and type of ferrofluid used.

The biologically inert carrier fluid can be water, which can act as a solvent for the ferromagnetic particles and/or viscosity enhancing agent. Alternatively, the biologically inert carrier fluid can be a buffer solution, such as a phosphate buffered saline (PBS) buffer, which can act as a solvent for the ferromagnetic particles and/or viscosity enhancing agent.

The present disclosure also provides a method of acquiring a direct visualization medical image of an internal structure. The method includes: a) introducing a ferrofluid into an area near the internal structure, thereby displacing a biological fluid within the area; b) acquiring the direct visualization medical image of the internal structure through the ferrofluid. The method can also include, prior to the acquiring of step b), contacting the internal structure with the ferrofluid. The internal structure can be any of the targets 150 described above. The introducing of step a) can be done via the channel 110 of the probe 100. The acquiring of step b) can be done via the optical waveguide 160 of the probe 100 and/or using the optical imaging system or OCT imaging system described herein. The contacting the internal structure with the ferrofluid can be achieved by moving the ferrofluid attractor 120 or by moving a distal tip or distal portion of the probe 100.

The systems, probes 100, and methods described herein can be used for any processes utilizing catheters, including flexible catheters. Such processes include in vivo imaging, such as in vivo cardiology or gastrointestinal tract imaging.

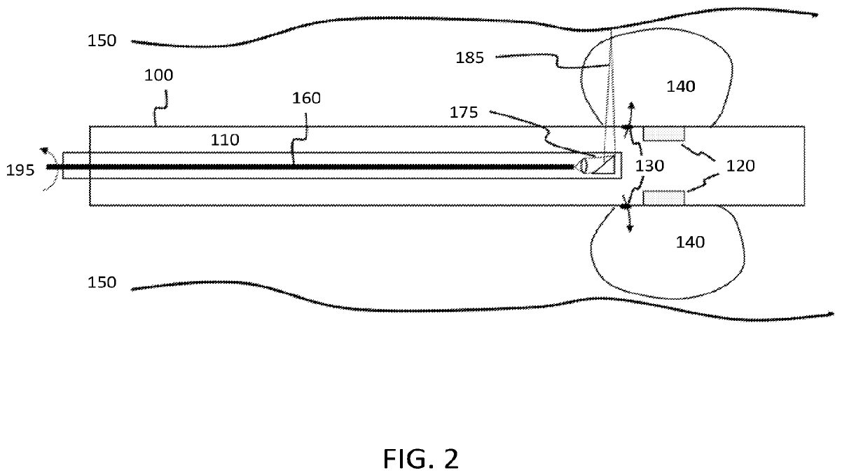

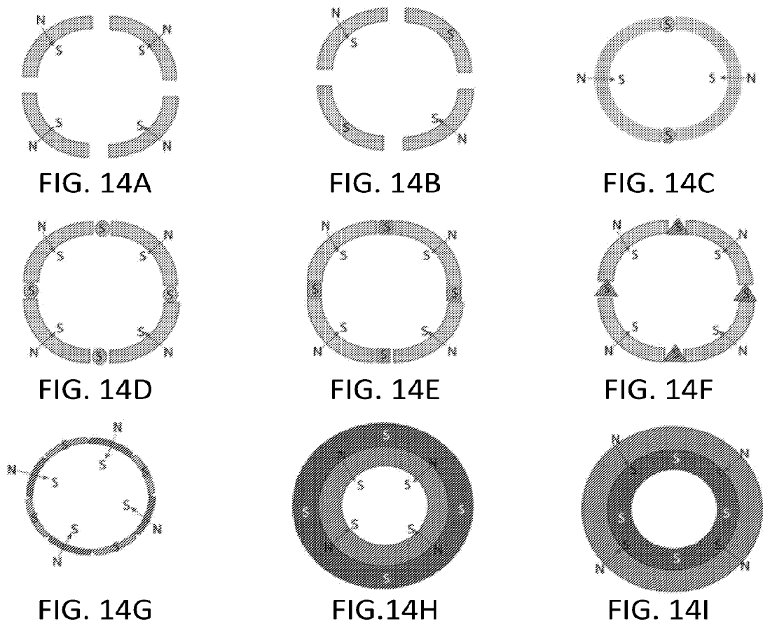

Referring to FIG. 2, an exemplary schematic of a probe 100 is illustrated. The probe 100 of FIG. 2 is illustrated in a configuration that is optimized for use in imaging circumferentially relative to the probe 100. The probe 100 includes a channel 110. The probe 100 includes a ferrofluid attractor 120 at a distal portion of the probe 100. The ferrofluid attractor 120 is arranged circumferentially relative to the distal portion of the probe 100 in order to retain the ferrofluid in a suitable location for circumferential imaging. Note that the configuration of the ferrofluid attractor 120 is merely an example, and the ferrofluid attractor 120 can be configured to have various other forms (e.g., as described below in connection with FIGS. 14A to 141, 15A to 15E, and 16A). The probe 100 includes two or more distal ports 130 in fluid communication with the channel 110. When introduced via the channel 110 and the two or more distal ports 130, the ferrofluid forms a ferrofluidic cloud 140 or multiple ferrofluidic clouds 140 due to being attracted by the ferrofluid attractor 120. The probe 100 is illustrated within a target 150 that is tubular in shape, such as a blood vessel. The probe 100 includes an optical waveguide 160 and an optional imaging optic 175 for coupling light 185 to the target 150. The light 185 is transmitted through the ferrofluidic cloud 140 to irradiate the target 150. Light returning from the target 150 traversed the ferrofluidic cloud 140 and is collected by the optical waveguide 160 or the optional imaging optic 175. The probe 100 can include a driveshaft 195 that is used to rotate the optical waveguide 160 and/or the optional imaging optic 175 to rotate the light 185 to provide circumferential irradiation to a substantially tubular target 150 and to acquire light returning in the same fashion. In some cases, the driveshaft 195 is excluded and the optional imaging optic 175 rotates via a motor located adjacent to the optional imaging optic 175.

The optical waveguide 160 can be an optical fiber, for example coupled to a laser emitting diode or other light source. The optical fiber can be a single-mode fiber. The optical fiber can be a double clad optical fiber. The optical waveguide 160 can serve as a sample arm for an OCT system.

The imaging optic 175 can be a lens, a reflector, other optics known to those having ordinary skill in the art to be useful for coupling light for the purposes of imaging, or combinations thereof. In some cases, the lens can be a ball lens, a spherical lens, an aspherical lens, a graded index (GRIN) fiber lens, an axicon, a diffractive lens, a meta lens, lensing with phase manipulation, or the like.

The driveshaft 195 can be coupled to the optical waveguide 160, the imaging optic 175, including a lens and/or a reflector, or a combination thereof.

The probe 100 can include a pump (not illustrated) for providing positive pressure to the ferrofluid when introducing the ferrofluid to the target and/or for providing negative pressure to remove the ferrofluid from the target.

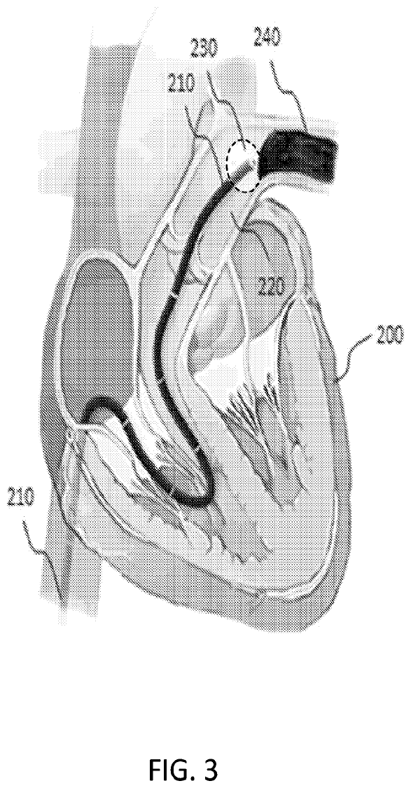

Referring to FIG. 3, one specific use of the probe 100 described above is illustrated. Specifically, the probe 100 is illustrated as being used to extract a blood clot from the pulmonary artery. As shown in FIG. 3, a flexible probe cardioscope 210 is inserted through the heart 200 into the pulmonary artery 220. The ferrofluid 230 (the extent of which is indicated by a dashed line) is transmitted at the tip of the cardioscope 210 and held into place by a magnet at the tip of the cardioscope 210 in a position where blood is displaced from the field of view of the imaging apparatus of the cardioscope 210. In the illustrated case, the cardioscope 210 is directed to a blood clot 240 within the pulmonary artery 220. An instrument such as a retrieval basket or a suction catheter can be inserted via the optional working channel 170 described above and can be used to remove the blood clot 240.

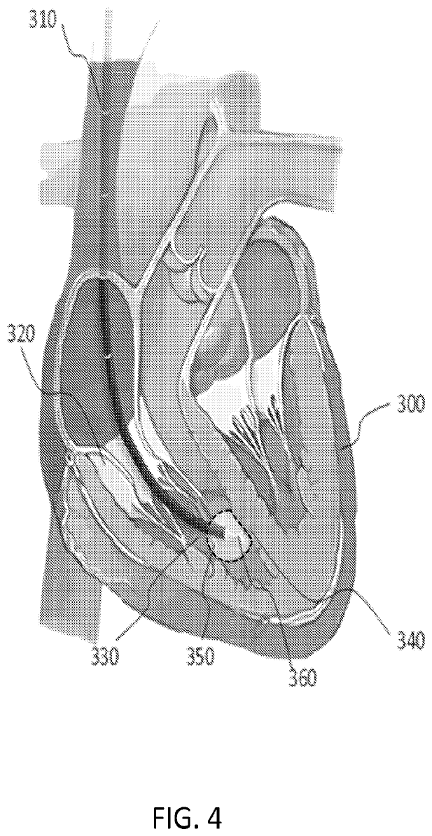

Referring to FIG. 4, another specific use of the probe 100 described above is illustrated. Specifically, the probe 100 is illustrated as being used to visualize and biopsy an endocardial surface. Referring to FIG. 4, a flexible probe cardioscope 310 is inserted through a tricuspid valve 320 of the heart 400. The tip of the cardioscope 310 is inserted into the right ventricle 330 in order to visualize the endocardial surface 340 for biopsy. The ferrofluid 350 is introduced from the tip of the cardioscope 310 and forms a cloud between the catheter and the cardiovascular structure, thereby displacing blood between the cardioscope 310 and the endocardial surface 340. Once a visual image is established, biopsy forceps 360 are introduced via the working channel 170 of the catheter 310 and are used to collect the tissue of the right ventricular wall 340 under direct visualization.

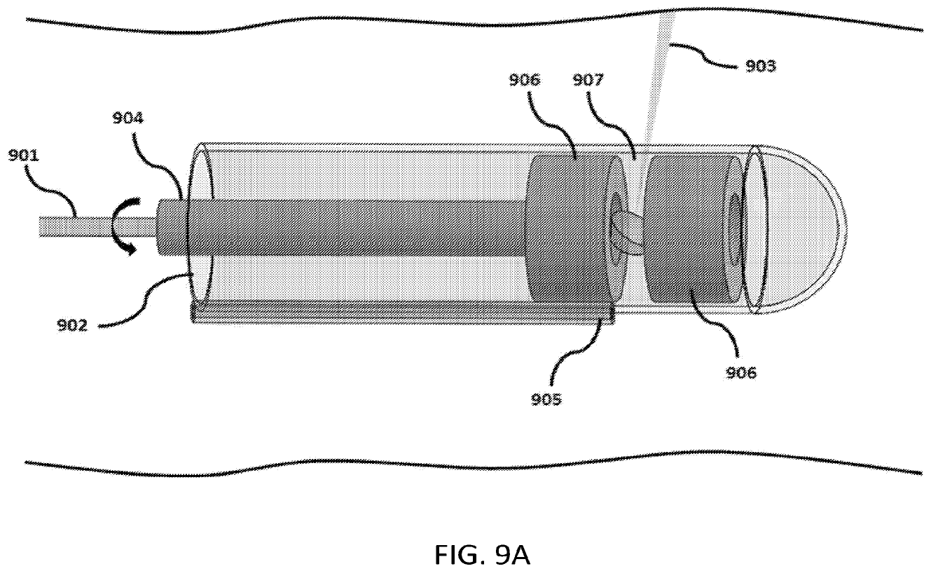

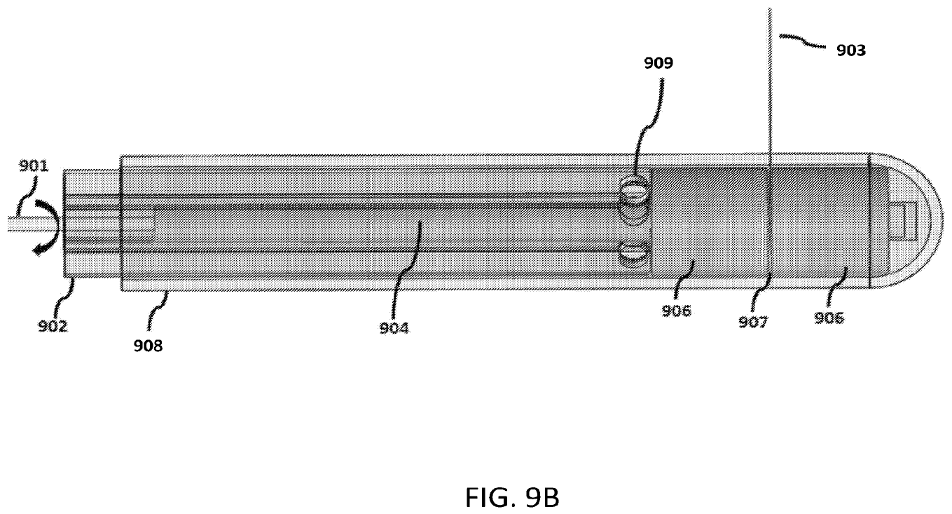

Referring to FIG. 9A, yet another schematic of a probe is shown. The probe of FIG. 9A is configured for circumferential imaging. An optical fiber 901 is housed in a drive shaft 904 which emits a beam 903 that rotates to acquire circumferential images. The beam 903 is emitted through a transmission gap 907 that lies between two magnets 906. The ferrofluid can be injected through a channel 905. Once injected, the ferrofluid would concentrate around the magnets 906 to displace surrounding fluid (e.g., blood). In the probe of FIG. 9A, a housing 902 can enclose magnets 906, and at least a portion of driveshaft 904. For example, housing 902 can be a catheter, or a capsule.

Referring to FIG. 9B, still another schematic of a probe is shown. The probe of FIG. 9B is configured for circumferential imaging, and is similar in some aspects to the probe of FIG. 9A in that the optical fiber 901 is housed in the drive shaft 904 which emits a beam 903 that rotates to acquire circumferential images, and the beam 903 is emitted through a transmission gap 907 that lies between two magnets 906. However, in the probe of FIG. 9B, the ferrofluid can be injected through a sheath 908 surrounding housing 902, and can flow out of sheath 908 via one or more openings 909. Once injected, the ferrofluid can concentrate around the magnets 906 to displace surrounding fluid (e.g., blood).

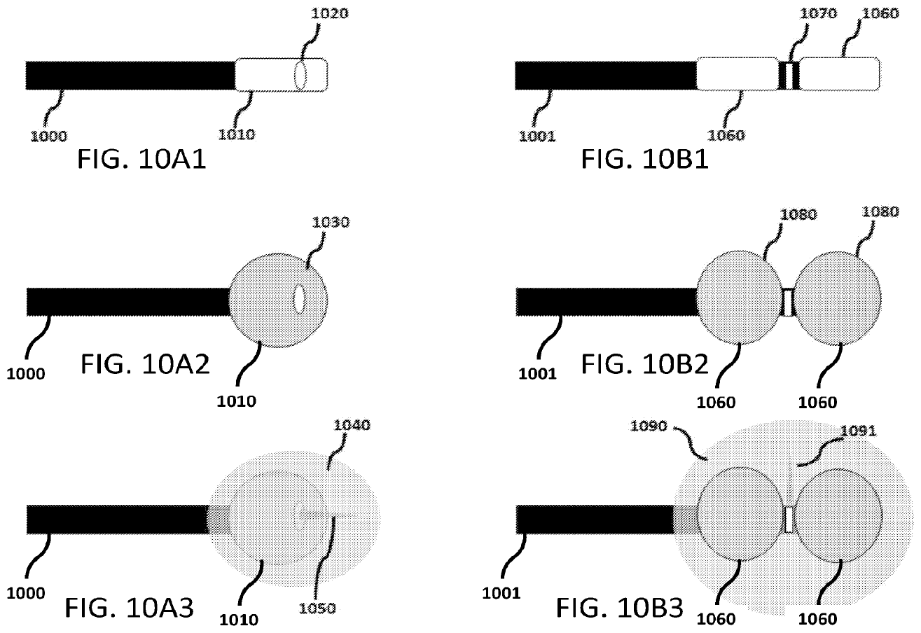

Referring to FIGS. 10A1 to 10B3, various schematics of probes are shown. FIG. 10A1 shows a catheter 1000 with an uninflated balloon 1010 at a distal end and forward-facing imaging tip 1020. FIG. 10A2 shows the balloon 110 inflated with iron particles 1030 in order to create a magnetic field at the tip of the catheter. For example, iron particles 1030 can be included in a ferrofluid, which can be the same ferrofluid that is used to produce a ferrofluid cloud, or a ferrofluid having different properties (e.g., a different concentration of particles, a different solvent, etc.). FIG. 10A3 shows a ferrofluid cloud 1040 that is injected outside of the catheter 1000 so that the particles concentrate around the magnetized balloon 1010. The ferrofluid cloud 1040 displaces surrounding biological fluid (e.g., blood) allowing light 1050 to be transmitted more easily toward a sample. FIG. 10B1 shows another catheter 1001 which includes two uninflated balloons 1060 on either side of a side viewing imaging tip 1070. FIG. 10B2 shows the balloons 1060 inflated with iron particles 1080 to create and/or augment a magnetic field at the tip of the catheter. FIG. 10B3 shows a ferrofluid cloud 1090 surrounding the magnetized balloons 1060. The ferrofluid cloud 1090 displaces surrounding biological fluid (e.g., blood) allowing light 1091 to be transmitted more easily toward a sample. The probe of FIGS. 10B1 to 10B3 can, for example, be used in connection with transcatheter procedures that involve navigation of the catheter through smaller vessels to reach a desired region in the heart.

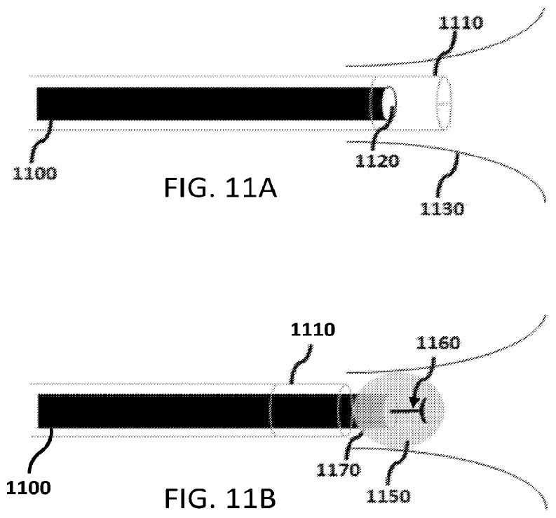

Referring to FIGS. 11A and 11B, additional schematics of probe are shown. FIG. 11A shows a catheter 1100 which includes a transparent outer sheath 1110 that extends beyond an imaging tip 1120 to allow for displacement of biological fluid (e.g. blood) and visualization. FIG. 11B shows the sheath 1110 being retracted from the tip. A ferrofluid cloud 1150 is shown concentrated around a magnetic tip 1170 of the catheter 1100. The ferrofluid cloud 1150 is able to displace the biological fluid (e.g., blood) previously displaced by the transparent sheath 1110. A bioptome 1160 is shown extending within the ferrofluid cloud 1150. The probe of FIGS. 11A and 11B can, for example, be used in connection with applications that involve navigating through regions of the heart with higher flow rates, as the probe of FIGS. 11A and 11B can facilitate direct visualization and the ability to work through the ferrofluid cloud in such an environment. Sheath 1110 can be deaired and/or flushed (e.g., with saline) prior to being inserted into a subject's cardiovascular system.

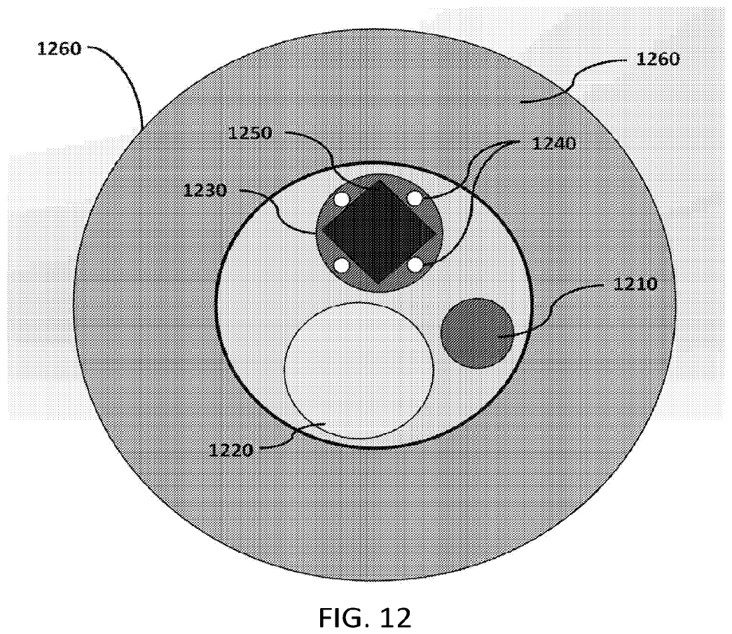

Referring to FIG. 12 a cross-section of a probe. FIG. 12 shows a probe 1200 that includes a ring magnet 1260 which can be magnetized through the diameter or thickness, or can include multiple arc segments (e.g., as described below in connection with FIGS. 14A to 14G) or multiple concentric rings (e.g., as described below in connection with FIGS. 14H and 14I). In the center of the magnets are three channels 1210, 1220, and 1230. The smaller channel 1210 can allow for ferrofluid injection, while channel 1220 can be a working channel that allows for insertion of instrumentation. The channel 1230 can include imaging components, which can include, for example, four multimode fiber bundles 1240 that can be used to illuminate a target and an optical sensor 1250. Note that this is merely an example, and other combination of optics can be used, such as optics described above in connection with FIGS. 1 and 2.

Referring to FIGS. 14A to 14I, cross-sections of various magnet configurations are shown. FIG. 14A shows a radial ring magnet that includes four arc magnets where north is on the outside and south is on the inside of each arc magnet. FIG. 14B shows four arc magnets where two are magnets are magnetized through the diameter and two are magnetized through the thickness (i.e., the two arc magnets shown as having the south pole exposed are magnetized through the thickness, such that the poles are aligned with the axial direction). FIG. 14C shows two arc magnets magnetized through the diameter with two rod magnets between them with south on the tip that is shown (i.e., the poles are aligned with the axial direction). FIGS. 14D, 14E, and 14F each show four arc magnets magnetized through the diameter with rod magnets (in 14D), rectangular magnets (in 14E), and pyramid magnets (in 14F) disposed between the ends of the arc magnets. FIG. 14G shows 8 arc magnets where four are magnetized through the diameter and four are magnetized through the thickness. FIG. 14H shows a radial ring inside a ring magnet that is magnetized through the thickness (i.e., the outer ring is magnetized such that the poles are aligned with the axial direction). FIG. 14I shows a ring magnet magnetized through the thickness with a radial ring magnet on the outside.

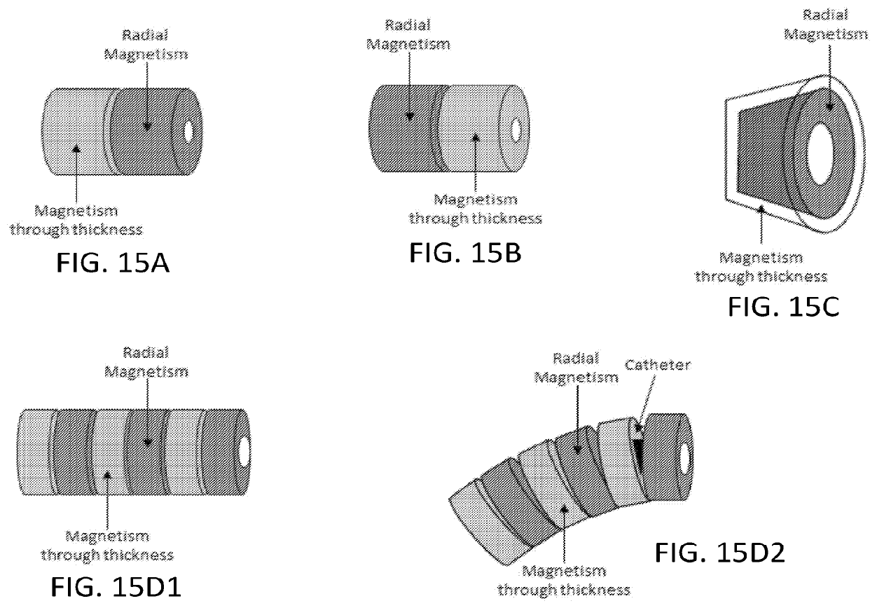

Referring to FIGS. 15A to 15E are additional examples of magnet configurations. FIG. 15A shows two ring magnets stacked, in which an imaging device can be inserted through the center of the ring magnets. For FIG. 15A, one magnet is magnetized radially while one magnet is magnetized through the thickness. For example, the radially magnetized ring magnet can be closer to the distal end of the probe (e.g., probe 100), while the axially magnetized ring magnet is farther from the distal end of the probe. FIG. 15B shows a similar configuration to FIG. 15A with the magnet order is reversed. FIG. 15C shows a funnel shaped configuration that includes two magnets, with an inner magnet radially magnetized, and an outer magnet axially magnetized through the thickness. In the configuration shown in FIG. 15C an imaging device can be inserted so that either the larger diameter end or the shorter diameter end of the funnel-shaped magnet can be closer to the distal end of the probe. FIG. 15D1 shows another magnet variation where radially magnetized magnets and magnets magnetized through the thickness are stacked along a length of the imaging device to increase the strength of the magnetic field at the tip of the device (e.g., the tip of the probe 100). Note that in addition to the configuration shown in FIG. 15D1 the magnets can be configured with various magnetizations. For example, all of the magnets in FIG. 15D1 can be radially magnetized or all the magnets can be magnetized through the thickness. FIG. 15D2 shows the magnet configuration in FIG. 15D1 arranged in a manner that facilitates bending of the imaging device (e.g., a catheter) along the length of the stacked the magnets, which can allow the number of magnets surrounding the tip of the probe to be increased, which can increase the magnetic field strength, while still allowing the device to bend. Configurations show in FIGS. 15A to 15E can augment the shape of the ferrofluid cloud to cause the cloud to extend more from the tip of the probe, rather than forming a sphere centered on the ferrofluidic attractor.

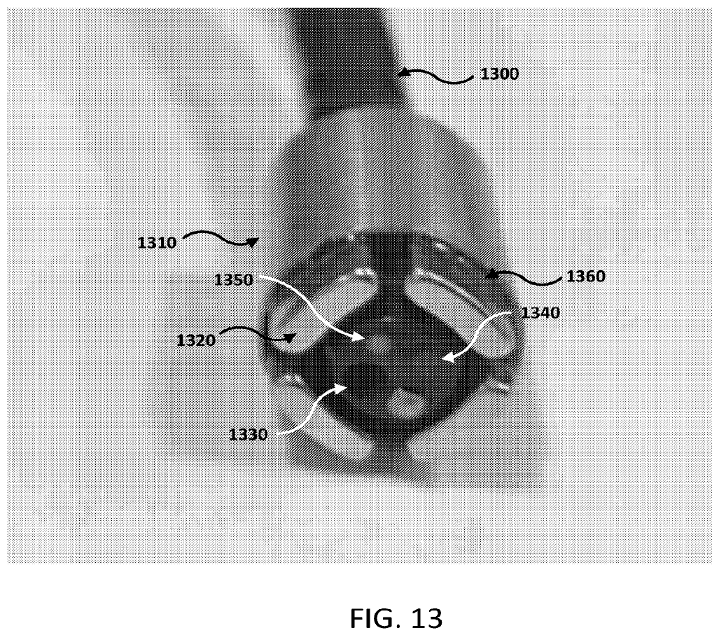

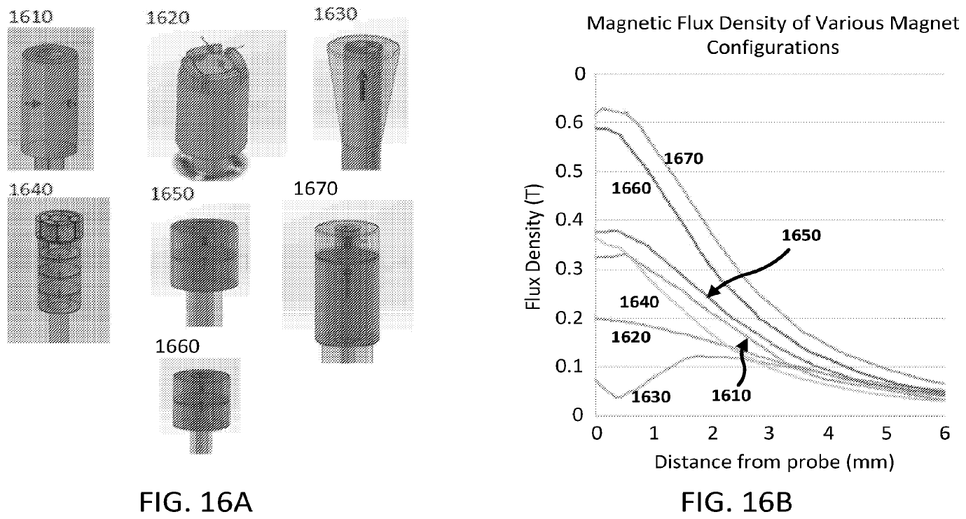

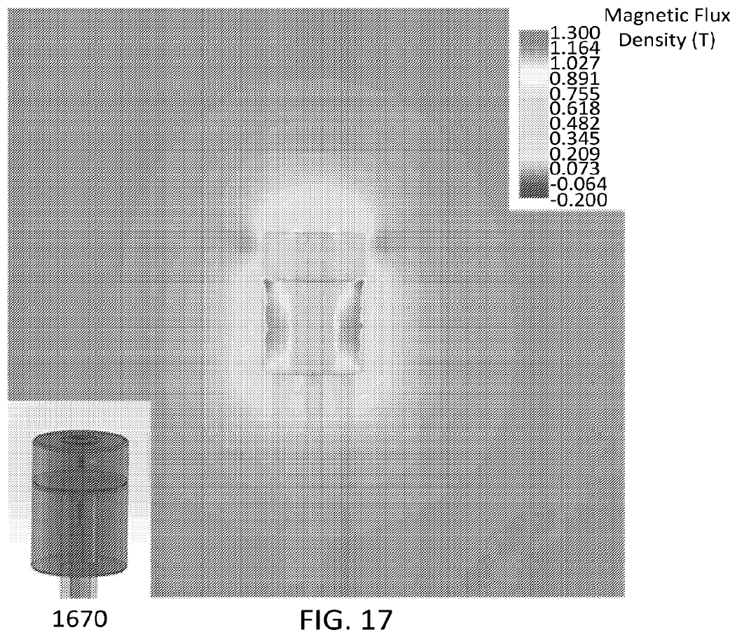

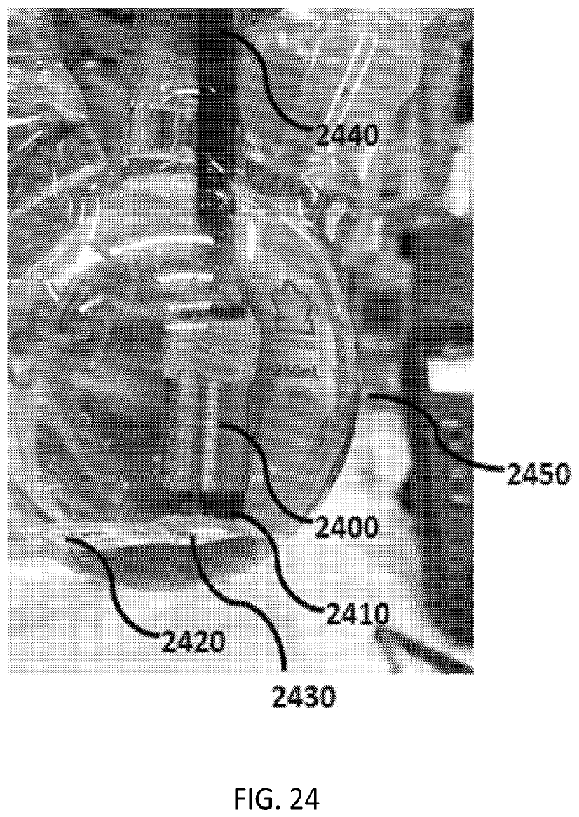

Referring to FIG. 16A, various magnet configurations and magnetic flux models are shown demonstrating a variety of potential magnet orientations, and FIG. 16B shows a depiction of flux density for each of the configurations in FIG. 16B as a function of distance from a tip of the probe extending toward a target. Configuration 1610 includes a radially polarized ring magnet with the north pole facing the inner diameter, and generates moderate magnetic flux (i.e., about 0.3 tesla (T) at the tip) that falls as the distance from the probe increases. Configuration 1620 includes four arcs polarized diametrically and encased by a stainless steel ring (note that this is a similar configuration to that depicted in FIG. 13), and generates less flux density at the tip (i.e. about 0.2 T at the tip). Configuration 1630 includes a cone magnet polarized axially, and generates even less flux density at the tip (i.e., less than 0.1 T). Configuration 1640 includes four axially polarized ring magnets and six arc magnets that are polarized diametrically and encased by a brass ring (e.g., note that this is a similar configuration to that depicted in FIG. 24), and generates higher flux close to the probe (i.e., about 0.36 T) that drops off relatively quickly. Configuration 1650 includes an axially polarized ring and a similarly-sized radially polarized ring nearer the distal end, and generates slightly denser flux along the entire profile than the configuration 1610 despite having similar radial dimensions and being shorter in the axial direction. Configuration 1670 includes an axially polarized ring magnet, and a radially polarized ring magnet nearer the distal end with both having a smaller inner diameter than the magnets shown in configurations 1610 and 1650. The configuration 1660 generates relatively higher density flux at the distal tip of the probe (i.e., about 0.59 T) and along the entire profile. The configuration 1670 is similar to the configuration 1660, but the axially polarized ring magnet is longer along the axial direction, and generates higher flux density near the tip (i.e., about 0.61 T) and along the entire profile than the configuration 1660. As shown in FIGS. 16A and 16B, combinations of axially and radially magnetized magnets can generate higher flux both close to and farther away from the probe than magnets having a single magnetization direction.

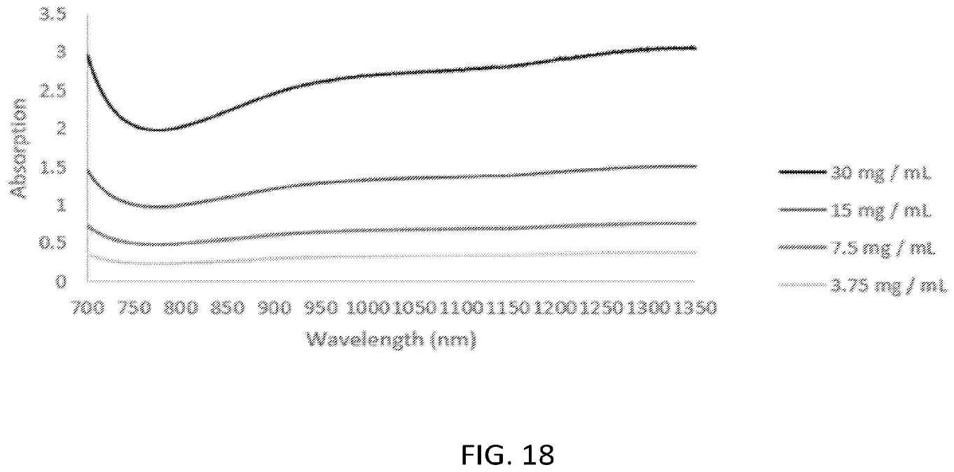

Referring to FIG. 17, a magnetic flux model corresponding to the configuration 1670 is shown. As described above, in the configuration 1670 two ring magnets are stacked, and a probe that includes channels for imaging, instrumentation, and/or in administration of ferrofluid can be inserted through the middle of the magnets. The distal magnet (the top magnet in FIG. 17) is magnetized radially with the north pole facing the inner diameter while the bottom magnet is magnetized axially through the thickness of the magnet with the north pole facing the distal tip and the radially magnetized ring magnet. The flux model shown in FIG. 17 is based on configuration 1670 with both magnets having an inner diameters of 4 mm and an outer diameters of 8 mm, the top magnet having an axial length of 4 mm, and the bottom magnet having an axial length of 8 mm. The flux density model shows a cross sectional cut through the center and gives indications of the shape and extent of ferrofluid cloud. Note that this is merely a particular example, and magnets having other dimensions can be used. For example, the magnet dimensions can be configured based on constraints on the probe, such as size and materials.

### EXAMPLES

The following examples set forth, in detail, ways in which the optical systems and/or the probes (e.g., the probe 100, the probes depicted in FIGS. 9A, 9B, 10A1 to 10B1, 11A, 11B, 12, 13, etc.), and/or the ferrofluid can be used or implemented, and will enable one of skill in the art to more readily understand the principles thereof. The following examples are presented by way of illustration and are not meant to be limiting in any way. Among other things, example 1 demonstrates that light can be transmitted through the ferrofluid; example 2 demonstrates that the ferrofluid can be concentrated using a magnetic field to form a ferrofluid cloud; example 3 demonstrates that the ferrofluid can displace blood; example 4 demonstrates that a ferrofluid cloud can displace blood and facilitate imaging (e.g., OCT imaging) of a target previously at least partially occluded by the blood; example 5 demonstrates an implementation of a scope that can be used in connection with ferrofluid imaging; example 6 demonstrates that light with a peak at about 775 nm can be transmitted through a ferrofluid that includes Feraheme; example 7 demonstrates that modifications of ferrofluid properties can impact how the ferrofluid affects light transmitted through the ferrofluid; example 8 demonstrates images captured using 400-1000 nm light and a Feraheme-based ferrofluid cloud in a blood-filled cavity; example 9 demonstrates images captured with and without a Feraheme-based ferrofluid cloud in an environment that simulates conditions in the right side of the heart using a pulsatile pump; example 10 demonstrates ferrofluid guided imaging used to directly visualization structures inside the right side of a blood-filled (non-beating) sheep heart; example 11 demonstrates of an instrument inserted through a ferrofluid cloud to biopsy tissue being imaged; example 12 demonstrates a probe inserted into a environment used to simulate conditions in the right side of the heart; example 13 demonstrates an example of a cardioscope that can be used for forward-facing ferrofluid imaging; example 14 demonstrates an example of an OCT probe that can be used for circumferential ferrofluid imaging; and example 15 demonstrates images captured through a Feraheme-based ferrofluid using OCT imaging techniques in a simulated coronary artery with constant blood flow.

### Example 1

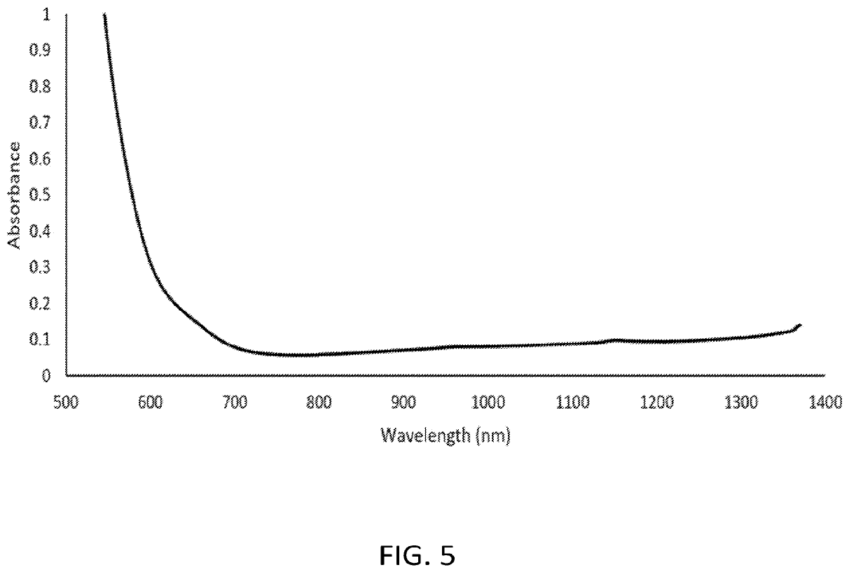

An exemplary ferrofluid for cardioscopy was prepared by mixing dextran-coated ferromagnetic particles having a mean diameter of 9 nm into PBS to provide a suspension. The average molecular weight of the dextran coating on the ferromagnetic particles was 40 kD. FIG. 5 shows the optical absorbance spectrum of this ferrofluid acquired using a 1-cm path length cuvette against an aqueous reference. The concentration of ferromagnetic nanoparticles in the solution was 0.8 mg/mL. The spectrum indicates a strong optical absorption below 600 nm due to the large absorption coefficient of the ferromagnetic nanoparticles. The spectrum also shows high optical transmission from 650 nm to 1400 nm, which can be used as an optical window to visualize an internal structure, as described elsewhere herein.

### Example 2

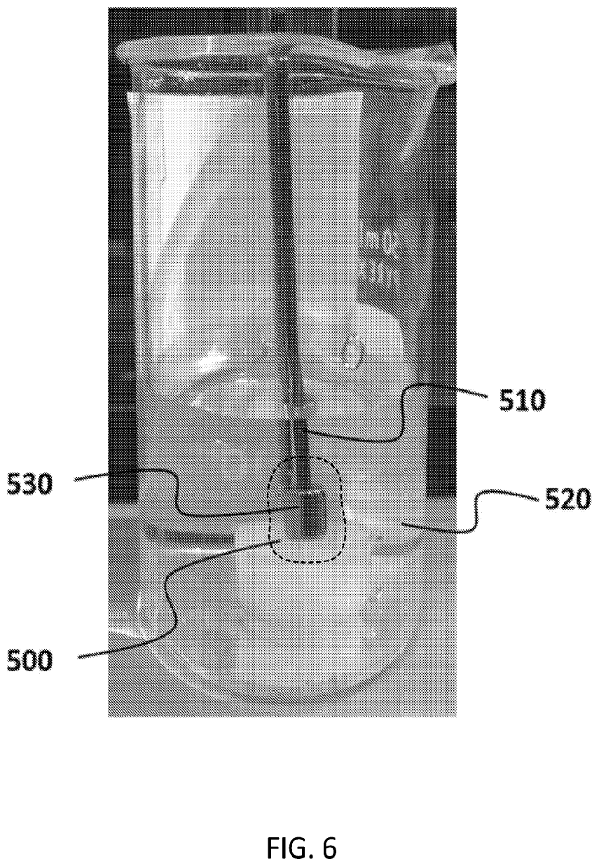

The ferrofluid of Example 1 was introduced via an optical probe having features described elsewhere herein into a PBS solution. Referring to FIG. 6, a photograph of a stable ferrofluid cloud 500 (the extent of which is indicated with a dashed line) formed around the base of an optical probe 510 placed in a PBS solution 520 is shown. A toroidal-shaped neodymium magnet 530, also referred to as a NdFeB magnet, was positioned at the base of the probe to generate the magnetic field that confines the ferromagnetic nanoparticles producing a roughly spherical cloud 500 having an approximate viewing depth of 3 mm beneath the optical probe. The magnet had an outer diameter of 4.67 mm and a length of 4.63 mm, thus providing the viewing depth of 3 mm.

### Example 3

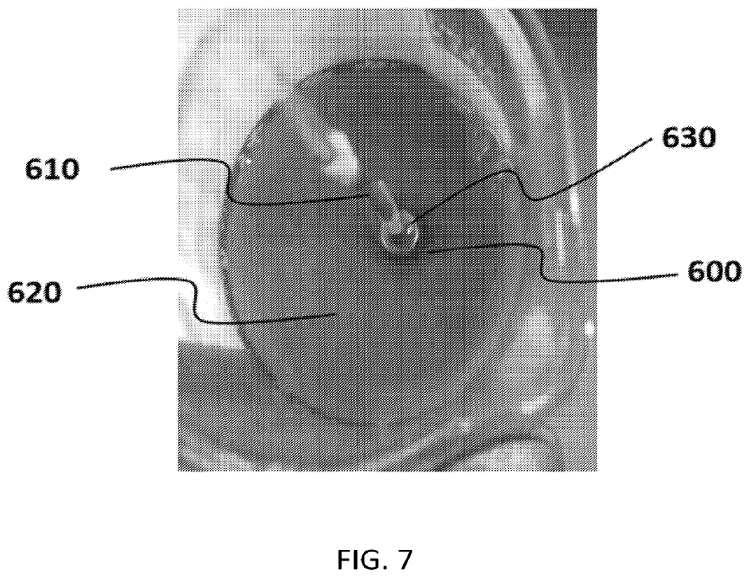

A ferrofluid having 40 kD dextran coated ferromagnetic nanoparticles in an amount of 0.4% (w/w) suspended in a 5% aqueous dextran solution was prepared and introduced into a blood sample via an optical probe having features described elsewhere herein. Referring to FIG. 7, a photograph shows a stable ferrofluid cloud 600 formed around an optical probe 610 in a whole blood 620 sample. At the distal tip of the optical probe 610, a toroidal NdFeB magnet 630 was positioned to generate the magnetic field. As the ferrofluid solution was delivered to the base of the magnet 630, the ferromagnetic particles were trapped by the strong magnetic field and displaced the surrounding whole blood 620, thereby creating an optical window 600 around the probe 610. The ferrofluid window 600 appears as a dark ring next to the probe 610 and NdFeB magnet 630. The addition of dextran assisted in the ability of the ferrofluid cloud 600 to displace the whole blood 620 and persist for several minutes.

### Example 4

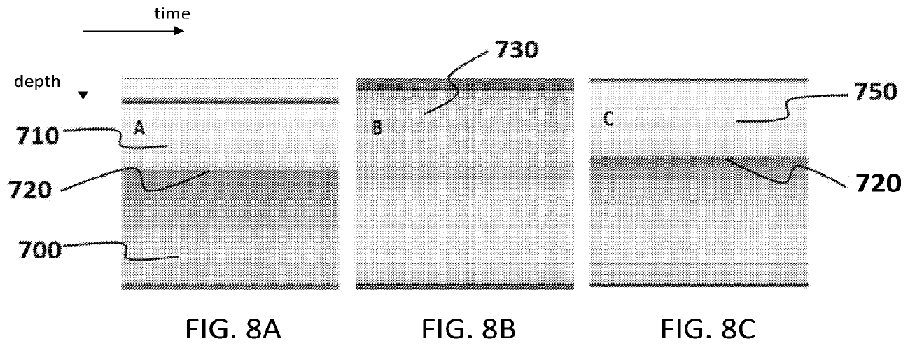

Referring to FIGS. 8A to 8C, a series of OCT images were collected from a probe, such as the probe 100 described above. OCT was conducted using light centered at 1310 nm with a 100 nm bandwidth. The OCT probe was fixed in space and did not scan. The vertical axis represents optical depth from the probe and the horizontal axis represents the time in which successive acquisitions of the OCT signal were recorded and processed into depth-resolved reflectivity profiles. Short horizontal lines in the images indicate particles that diffused into and out of the OCT beam while long horizontal lines indicate reflections from stationary structures. Referring to FIG. 8A, an image of a stationary nylon target 700 imaged through saline 710 is shown. The presence of inclusions within the nylon target 700 is shown below a target interface 720 between the saline 710 and the nylon target 700. Referring to FIG. 8B, an image of the same stationary nylon target is shown imaged through heparinized whole blood 730 without a ferrofluid cloud. Strong scattering from the red blood cells and hematocrit in the whole blood obscure the target interface and significantly limit visibility. Referring to FIG. 8C, an image of the same stationary nylon target in whole blood is shown imaged through a ferrofluid cloud 750 introduced into the whole blood. The target interface 720 is much more clearly visible when the ferrofluid cloud 750 is used. The ferrofluid cloud 750 remained stable for several minutes. The ferrofluid used in FIG. 8C is the ferrofluid of Example 3.

### Example 5

Referring to FIG. 13, a photograph of a magnet on an Olympus GIF type XP160 Evis Exera Gastrointestinal videoscope 1300. Surrounding the magnet is a thin stainless steel casing 1310. There are four arc magnets 1320 which are radially magnetized through the diameter with south on the inner diameter allowing the field lines to congregate at the center. There is a thin layer of epoxy 1360 coating the lip between the stainless steel casing 1310 and the magnets 1320. The scope includes a working channel 1330, sensor 1340, and light source 1350.

### Example 6

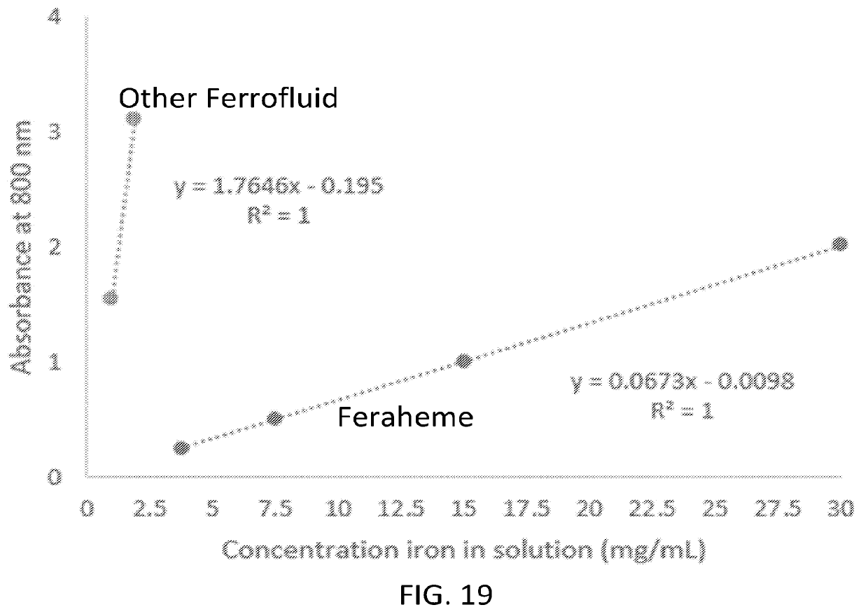

Referring to FIG. 18, optical absorbance spectra of Feraheme, a clinically approved ferrofluid, acquired using a 1-cm path length cuvette against an aqueous reference is shown. The absorbance of Feraheme at a clinical concentration of 30 mg Fe/mL, and diluted to 15, 7.5, and 3.75 mg Fe/mL is shown for light from 700 to 1350 nm. The results indicate that the absorbance has a minimum at around 775 nm in the range of wavelengths shown. This suggests that using a light source that extends beyond visible spectrum (400-750 nm) into the near-infrared may improve visualization through Feraheme.

### Example 7

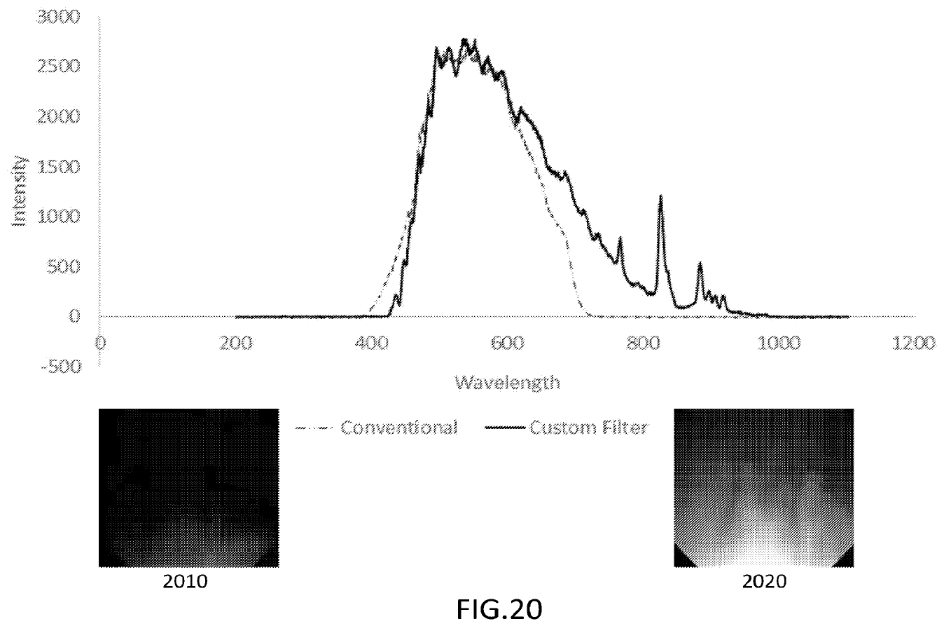

Referring to FIG. 19, the optical absorbance of Feraheme at 800 nm is shown with optical absorbance of a different non-clinical ferrofluid from Ferrotec. Absorbance is shown for the two ferrofluids at multiple iron concentrations. The results indicate that the non-clinical ferrofluid has a higher absorbance at 800 nm than Feraheme. When comparing the two ferrofluids, the size of the nanoparticles for the non-clinical ferrofluid were smaller than for the Feraheme. Also, the carbohydrate coating on the nanoparticles differed. Both ferrofluids were tested in a pulsatile pump, and the non-clinical ferrofluid maintained a shape of the ferrofluid cloud at larger flow rates and larger pressure than Feraheme at the same iron concentration. These result demonstrate that various properties of the ferrofluid affect the ability of the ferrofluid to transmit light and withstand flow (e.g., of blood). This also suggests that different ferrofluids can be used for different clinical applications when certain properties are desired.

### Example 8

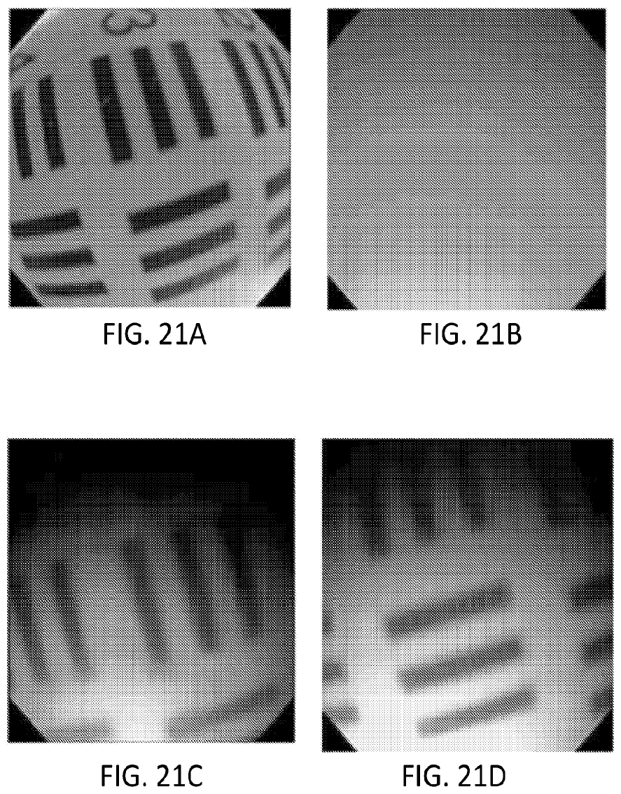

Referring to FIG. 20A, a light spectrum is shown. The dashed line corresponds to conventional white light imaging (e.g., 400-750 nm light), and the solid line corresponds to the usage of a filter in the light source that results in imaging that incorporates the near-infrared in addition to visible light (e.g., 400-1000 nm). An image 2010 was acquired using 400-750 nm light to image sheep heart issue in a blood-filled cavity, while an image 2020 was acquired using 400-1000 nm light to image the sheep heart tissue at the same position. Image 2020 demonstrates that by incorporating near-infrared light, increased detail of the sheep heart tissue is discerned. Image 2020 demonstrates that different wavelengths of light can be used for different clinical applications through specific ferrofluids.

### Example 9

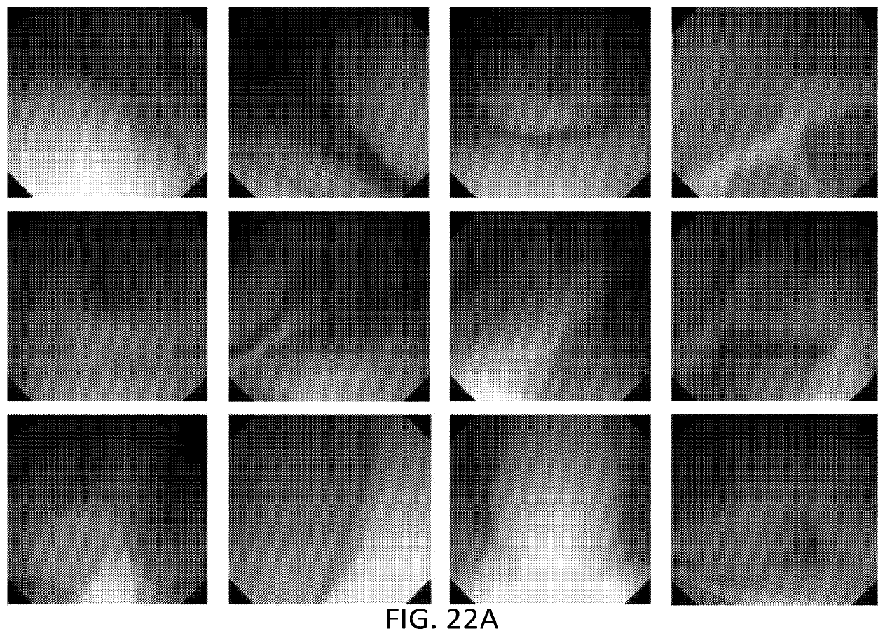

Referring to FIGS. 21A to 21D, a series of images recorded using the Olympus GIF type XP160 Evis Exera Gastrointestinal videoscope is shown (note that this corresponds to the probe described above in connection with FIG. 13). The images were recorded in a pulsatile pump (e.g., as described below in connection with Example 12 and FIG. 24) simulating the flow rates, pressure, and temperature of the right side of the heart at distance from the target of about 4 mm. Referring to FIG. 21A, an image of a USAF Field target is shown through water. Referring to FIG. 21B, an image of the same USAF Field Target is shown covered by blood in the absence of a ferrofluid cloud disallowing visualization by the videoscope. Referring to FIG. 21C, an image of the same USAF Field target is shown in blood in the presence of a Feraheme cloud as described herein displacing the surrounding blood and allowing visualization of the target. The image in FIG. 21C was recorded using conventional white light imaging. Referring to FIG. 21D, the same target is shown in blood displaced by Feraheme using white light and near-infrared imaging (400-1000 nm). The ferrofluid attractor was a radial ring magnet (e.g., as shown in FIG. 13). These images demonstrate the ability of the clinically approved ferrofluid to displace blood in a pulsatile pump while allowing imaging through the ferrofluid cloud.

### Example 10

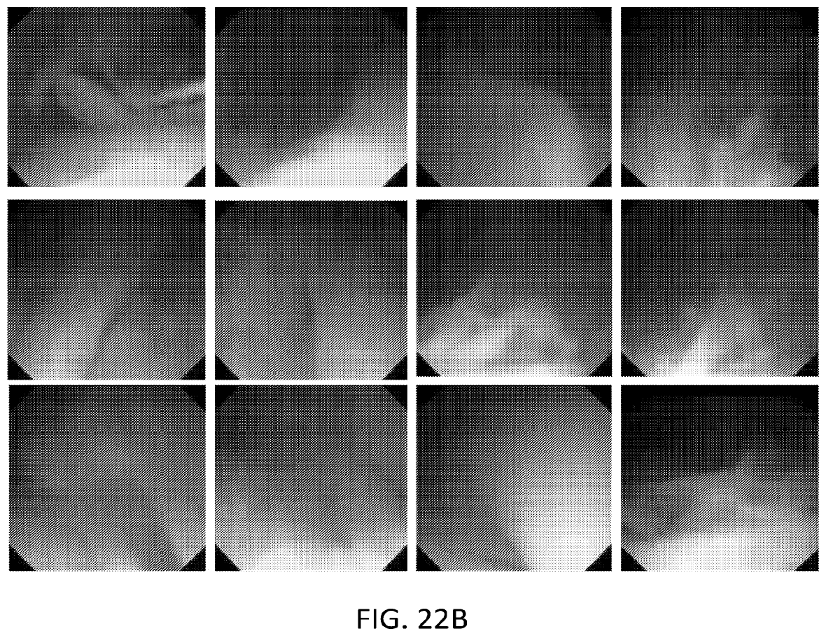

Referring to FIG. 22A and 22B, various still shots are shown of Feraheme imaging inside of a blood-filled sheep heart. The imaging demonstrates the ability of the ferrofluid guided imaging to allow for direct visualization of significant structures inside the major chambers of the heart. The same imaging system as described in Example 9 was used to capture the images.

### Example 11

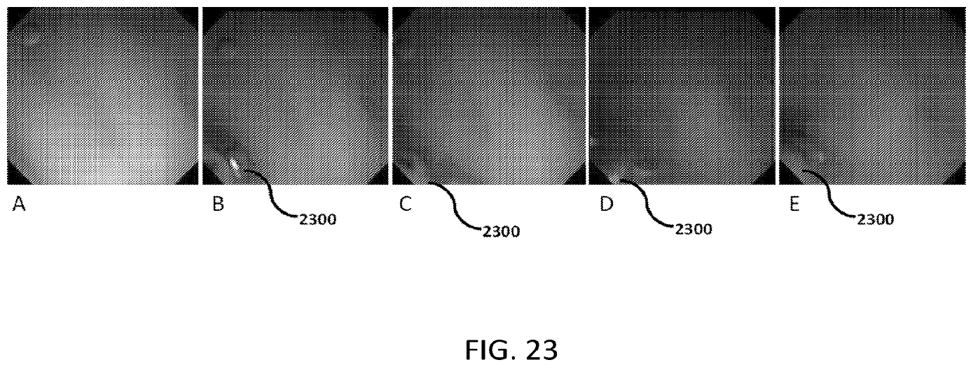

Referring to FIG. 23, a sequence of still shots are shown of Feraheme imaging inside a blood-filled sheep heart. A bioptome 2300 is shown protruding through the ferrofluid cloud and successfully collecting a tissue sample from within the heart guided by direct visualization. The images demonstrate the ability of the ferrofluid guided imaging to allow instrumentation through the cloud during direct visualization. The same imaging system as described in Example 9 was used.

### Example 12

Referring to FIG. 24, a photograph demonstrating a pulsatile pump used to generate the images in FIGS. 21A to 21D is shown. The pulsatile pump simulates the flow rate, pressure, and temperature of the heart. The photograph shows the endoscope 2440 with a radial ring magnet 2400 attached. The ferrofluid cloud 2410 can be seen protruding past the endoscope and the light transmitting through the ferrofluid cloud 2410 can be identified. Below the scope is the USAF target 2420. The photograph shows the flask 2450 filled with saline. The pump was filled with blood in order to test the ability of the ferrofluid to withstand various flow rates and pressure while still allowing visualization through blood. Images acquired when the pump was filled with blood and saline are described above in connection with FIGS. 21A to 21D.

### Example 13

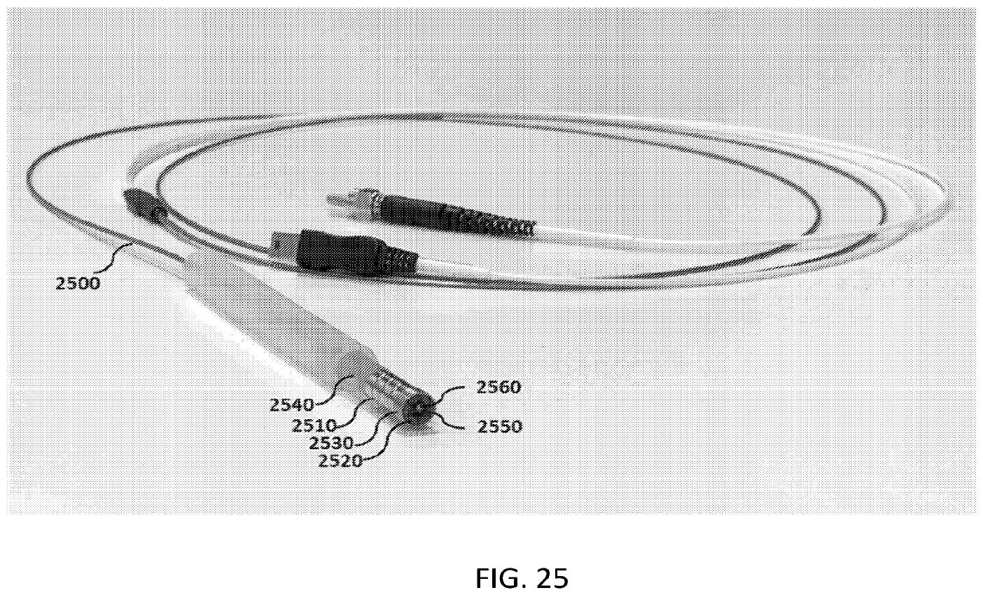

Referring to FIG. 25, a photograph of a smaller probe using an Enable Imaging minnieScope-XS miniature videoscope 2500 is shown. The scope can be inserted through a polymer tubing with two channels. One channel includes the scope and the second channel 2540 allows ferrofluid injection or instrumentation. A combination of magnets surrounds the polymer tubing, and includes four ring magnets 2510 magnetized through the thickness with north on the distal end and six arc magnets 2520 which are magnetized through the diameter with north on the inner diameter. Surrounding the arc magnets is a thin brass encasing 2530. This configuration can cause the magnetic field lines to aggregate near the center of the probe, and can cause the magnetic field lines to project forward. The scope 2500 itself includes optical fiber bundle waveguides 2550 for target illumination and a sensor 2560. The outer diameter of the scope is 1.7 mm while the total diameter of the probe is 7 mm, which allows for navigation into smaller areas of inside the heart.

### Example 14

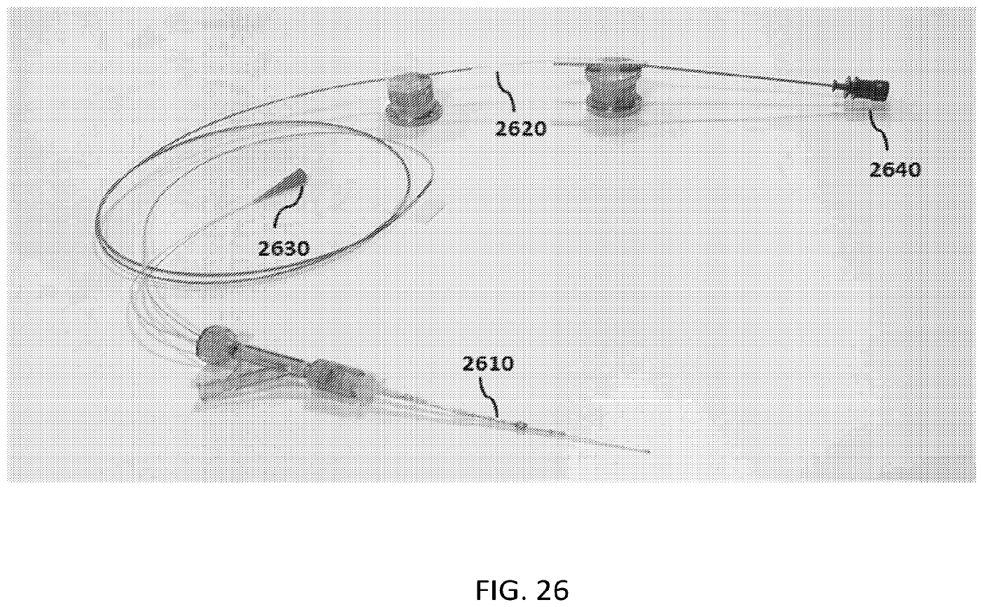

Referring to FIG. 26, a photograph of a probe configured for circumferential imaging is shown. A drive shaft 2620 houses an optical fiber that emits a beam from a ball lens and through the two ring magnets 2610. The drive shaft can be connected to a rotary junction using a connection 2640. Together, the drive shaft 2620, optical fiber, and ball lens can rotate 360 degrees to acquire circumferential images. Ferrofluid can be injected through an injection site 2630 to concentrate around the ring magnets 2610 and displace blood.

### Example 15

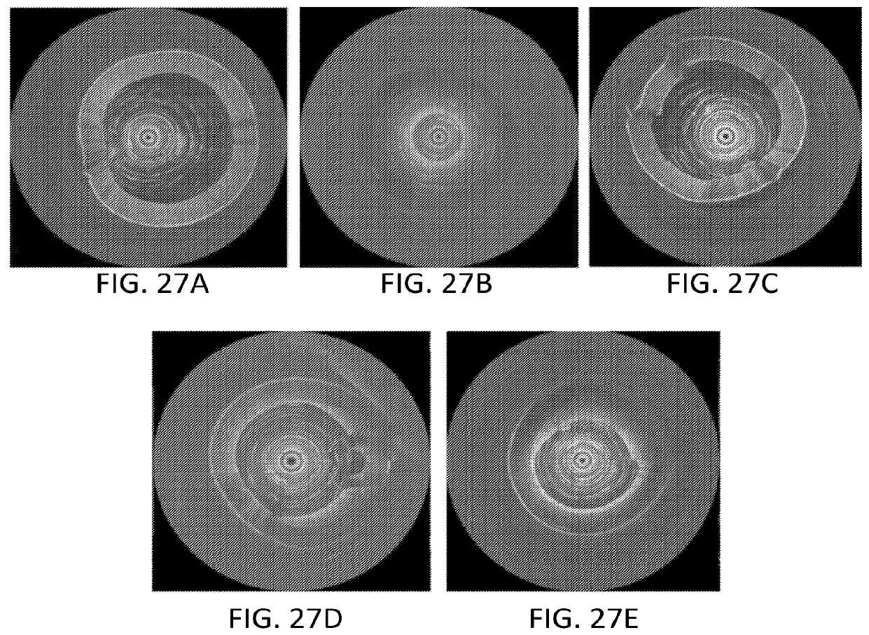

Referring to FIGS. 27A to 27E, a series of images recorded using an OCT probe for circumferential imaging (i.e., the system described above in connection with FIG. 26) is shown. Circumferential OCT imaging was conducted using light centered at 1310 nm with a 100 nm bandwidth. The images were recorded with the probe inserted into a nylon tube to simulate a coronary artery and allowed for flow of various liquids. The nylon tube was imaged as the target at 1.5 mm and had a thickness of 0.7 mm. Referring to FIG. 27A, a clear OCT image of the cross section of the nylon tube is shown through water. Referring to FIG. 27B, the nylon tube was filled with blood at static conditions which disallowed visualization by the OCT probe. Referring to FIG. 27C, an image of the nylon tube with blood at static conditions is shown with a Feraheme cloud displacing the surrounding blood and allowing clear visualization of the entire thickness of the target. Referring to FIG. 27D, an image of the nylon tube is shown with continuous Feraheme injection and blood flow through the nylon tube simulating flow in the coronary artery. These images demonstrate the ability of clinically approved ferrofluid to displace blood in a stimulated artery with constant flow to allow circumferential imaging through the ferrofluid cloud.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto. Indeed, the arrangements, systems, and methods according to the exemplary embodiments of the present disclosure can be used with and/or implemented any OCT system, OFDI system, SD-OCT system or other imaging systems capable of imaging in vivo or fresh tissues, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, U.S. Patent Publication No. 2002/0122246, published on May 9, 2002, U.S. Patent Application 61/649,546, U.S. patent application Ser. No. 11/625,135, and U.S. Patent Application 61/589,083, the disclosures of which are incorporated by reference herein in their entireties. The entire disclosure of each patent and publication cited herein is incorporated by reference, as if each such patent or publication were individually incorporated by reference herein.

1. A probe having a proximal portion and a distal portion, the probe comprising:
a channel having a proximal port positioned at the proximal portion of the probe, a distal port positioned at the distal portion of the probe, the proximal port, and the distal port having size dimensions that allow a ferrofluid to enter the channel via the proximal port, move along the channel, and exit the channel via the distal port; and
a ferrofluid attractor coupled to the distal portion of the probe, the ferrofluid attractor having magnetic properties and positioning relative to the distal port to magnetically attract the ferrofluid when exiting the distal port.

2. The probe of claim 1, wherein the ferrofluid attractor is a magnet and the magnetic properties include a magnetism sufficient to magnetically attract the ferrofluid.

3. The probe of claim 2, wherein the magnet is configured to be adjustable between a first state associated with a first magnetic flux at the distal port and a second state associated with a second magnetic flux at the distal port that is lower than the first magnetic field strength.

4. The probe of claim 3, wherein the magnet is coupled to an actuator configured to move the magnet relative to the distal port or a distal surface of the distal portion of the probe.

5-8. (canceled)

9. The probe of claim 1, wherein the ferrofluid attractor is circumferentially arranged relative to an external distal portion surface of the distal portion of the probe.

10. (canceled)

11. The probe of claim 9, wherein the ferrofluid attractor comprises a plurality of attractor components,
the plurality of attractor components comprising:
a first ring magnet and a second ring magnet, wherein the first ring magnet has poles aligned with a longitudinal axis of the probe and the second ring magnet has poles aligned orthogonally to the longitudinal axis of the probe; or
a first arc magnet and a second arc magnet, wherein the first arc magnet has poles aligned orthogonally to a longitudinal axis of the probe.

12. (canceled)

13. (canceled)

14. The probe of claim 11, wherein the second arc magnet has poles aligned with the longitudinal axis of the probe.

15. The probe of claim 9, wherein the ferrofluid attractor comprises a balloon configured to be inflated by a fluid comprising ferromagnetic particles.

16. The probe of claim 1, the channel further comprising one or more additional distal ports positioned at the distal portion of the probe.

17. The probe of claim 16, wherein the distal port and the one or more additional distal ports are arranged circumferentially about the distal portion of the probe.

18. (canceled)

19. The probe of claim 1, the probe further comprising a working channel having a working channel proximal opening positioned at the proximal portion of the probe and a working channel distal opening positioned at the distal portion of the probe.

20-24. (canceled)

25. The probe of claim 1, the probe further comprising:
a lens coupled to the distal waveguide end, wherein the lens includes a reflective surface configured to direct light emerging from the optical waveguide to a target
the probe further comprising a driveshaft, wherein the optical waveguide, the lens, or a combination thereof is coupled to the driveshaft.

26-36. (canceled)

37. A method of acquiring a direct visualization medical image of an internal structure, the method comprising:
a) providing a probe having a proximal portion and a distal portion, the probe comprising:
a channel having a proximal port positioned at the proximal portion of the probe, a distal port positioned at the distal portion of the probe, the channel, the proximal port, and the distal port and
a ferrofluid attractor coupled to the distal portion of the probe, the ferrofluid attractor having magnetic properties and positioning relative to the distal port to magnetically attract the ferrofluid when exiting the distal port;
b) introducing a ferrofluid into an area near the internal structure via the channel of the probe, thereby displacing a biological fluid within the area, the ferrofluid retained in the area using a magnetic effect; and
c) acquiring the direct visualization medical image of the internal structure through the ferrofluid.

38. The method of claim 37, the method further comprising, prior to the acquiring of step b), contacting the internal structure with the ferrofluid.

39. (canceled)

40. The method of claim 38, wherein the contacting is achieved by moving the ferrofluid attractor of the probe.

41. The method of claim 38, wherein the contacting is achieved by moving a distal tip of the probe

42. (canceled)

43. The probe of claim 1, further comprising:
an optical waveguide having a proximal waveguide portion positioned at the proximal portion of the probe and a distal waveguide end positioned at the distal portion of the probe.

44. The probe of claim 1, wherein the channel has an interior surface composed of a material that is chemically and magnetically inert to the ferrofluid.

45. The probe of claim 1, wherein the size dimensions of the proximal port and the distal port allow the ferrofluid to enter the channel via the proximal port, move along the channel, and exit the channel via the distal port when the ferrofluid is introduced at a positive pressure.

46. The probe of claim 1, further comprising:
an image sensor positioned at a distal portion of the probe, and
a light source configured to illuminate a target at a distal end of the probe.
